(19) United States
(12) Patent Application Publication
(10) Pub. No.: US 2005/0014689 A1
(43) Pub. Date: Jan. 20, 2005

(54) Remedies for life style-related diseases or cibophobia and method of screening the same

(76) Inventors: Eiji Sugaru, Osaka-shi (JP); Atsushi Tsuchida, Nara-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/491,654

(22) PCT Filed: Oct. 2, 2002
(86) PCT No.: PCT/JP02/10250

(30) Foreign Application Priority Data
Oct. 2, 2001 (JP) 2001-306872

(51) Int. Cl. A61K 48/00; A61K 39/395; A61K 38/17

(52) U.S. Cl. 514/12; 514/44; 424/145.1

(57) ABSTRACT

The present invention provides a novel therapeutic agent for a lifestyle-related disease or cibophobia, which is superior in controlling food intake, and a screening method therefor. More specifically, a therapeutic agent for a lifestyle-related disease containing, as an active ingredient, a substance that suppresses expression or function of GPCR that expresses in hypothalamus, clone 901, a therapeutic agent for cibophobia containing, as an active ingredient, a substance that enhances expression or function of the clone, a screening system consisting of a series of coexpression systems of clone 901 and various G proteins, and a screening method for a substance having a therapeutic activity against a lifestyle-related disease or cibophobia, which includes use of the screening system, are provided.

### TECHNICAL FIELD

The present invention relates to a therapeutic agent for a lifestyle-related disease, which comprises, as an active ingredient, a substance that suppresses expression or function of orphan GPCR that expresses in hypothalamus. More particularly, the present invention relates to a therapeutic agent for a lifestyle-related disease, which comprises, as an active ingredient, antisense nucleic acid of GPCR mRNA, an expression vector containing the nucleic acid or a host cell transfected with the expression vector, or a therapeutic agent for a lifestyle-related disease, which comprises, as an active ingredient, a substance having an antagonist activity to GPCR. Furthermore, the present invention relates to a coexpression system of a GPCR and a G protein and a screening method for a therapeutically active compound for a lifestyle-related disease using the same. The present invention moreover relates to a therapeutic agent for cibophobia, which comprises, as an active ingredient, a substance that enhances the expression or function of GPCR. In detail, the present invention relates to a therapeutic agent for cibophobia, which comprises, as an active ingredient, a substance having an agonist activity to GPCR. Furthermore, the present invention relates to a coexpression system of a GPCR and a G protein and a screening method for a therapeutically active compound for cibophobia using the same.

### BACKGROUND ART

Due to westernization of the eating habits, increase of social stress and the like in recent years, the number of patients with obesity and accompanying lifestyle-related diseases, particularly type II diabetes, has been increasing dramatically. For the therapy of these cases, exercise therapy and diet therapy are performed first. When the weight control is insufficient even by these treatments, drug therapy is performed. In doing so, a therapeutic agent which is superior in controlling food intake, body weight and blood glucose, and which is safe has been desired.

On the other hand, stress society of the present age combined with epidemic of an easy diet has brought a rapid increase of psychogenic eating disorder, such as cibophobia, in adolescent women. While it is indispensable to solve psychological problems to treat these diseases fundamentally, drug therapy may be performed to forcibly control feeding behavior as a supportive therapy. A therapeutic agent in this case is also requested to be able to promote eating while controlling body weight and glucose level.

Eating is mainly controlled by the central nervous system, and many nervous systems ruling over instinctive behaviors of human, such as appetite and the like, are located particularly in hypothalamus. In fact, when hypothalamus ventromedial nucleus of rat is damaged, it causes overeating and obesity, whereas when hypothalamus lateral nucleus is damaged, feeding behavior is not taken. In addition, localization of receptors of leptin and neuropeptides (e.g., neuropeptide Y (NPY)), which are involved in eating control, has been shown heretofore, which makes it clear that hypothalamus is an important organ for feeding behavior.

It has become clear that receptors of physiologically active substances in the central nervous system including hypothalamus, particularly G protein-coupled receptor (GPCR), correlates with feeding behavior. For example, it is known that knockout (KO) mouse with serotonin 5-THT₂C receptor suffers from chronic overeating. In addition, melanocortin 4 receptor antagonist increases food intake and, on the contrary, NPY Y5 receptor antagonist suppresses food intake.

Thus, stimulation of the nervous system in hypothalamus is considered to influence the feeding behavior, and substitute operation of signal transduction through GPCR, which expresses in hypothalamus, by the use of a low molecular weight compound meets the above-mentioned object of controlling food intake, body weight, glucose level and the like. However, a drug having such an action mechanism has not been marketed at present, and development of such a pharmaceutical agent has been highly desired.

It is therefore an object of the present invention to regulate feeding behavior by allowing an external factor, particularly a factor that suppresses or promotes expression or function of GPCR involved in the feeding behavior, to function, thereby providing a means for treating a lifestyle-related disease mainly caused by overeating or obesity, or cibophobia. It is another object of the present invention to provide a compound having a controlling effect on eating disorder such as overeating or apocleisis, obesity and the like, and a screening method for such a compound.

### DISCLOSURE OF THE INVENTION

To achieve the above-mentioned objects, the present inventors first searched a gene encoding a receptor expressed in hypothalamus from a database, and, as a result, found a certain orphan GPCR (hereinafter to be referred to as clone 901). Next, the present inventors administered an antisense oligo DNA of mRNA encoding this receptor to test animals to inhibit its expression. As a result, food intake was in fact suppressed. Therefrom it has been clarified that clone 901 is a receptor involved in the signal transduction which positively regulates the feeding behavior, and therefore, a substance inhibiting expression or function of this receptor shows a therapeutic effect on lifestyle-related diseases including type II diabetes caused by overeating, obesity and the like. In contrast, a substance enhancing expression or function of this receptor should show a therapeutic effect on eating disorders such as cibophobia. Thus, the present inventors constructed a series of coexpression systems of clone 901 and various G proteins, and using same, searched for an agonist or an antagonist to clone 901, based on which developed a method for screening for a compound having a therapeutic activity against lifestyle-related diseases or cibophobia, which resulted in the completion of the present invention.

Accordingly, the present invention provides a therapeutic agent for a lifestyle-related disease, which comprises, as an active ingredient, a substance that suppresses expression or function of clone 901, a GPCR expressed in hypothalamus. As a substance capable of specifically suppressing the expression of clone 901, an antisense nucleic acid of mRNA encoding clone 901 can be preferably mentioned. In this case, the antisense nucleic acid can be provided not only as it is but in the form of an expression vector encoding said nucleic acid or a host cell into which said expression vector has been introduced. In addition, as a substance capable of specifically suppressing the function of clone 901, an antagonist to said receptor can be mentioned.

The present invention further provides a therapeutic agent for cibophobia, which comprises, as an active ingredient, a substance enhancing expression or function of clone 901. As a substance capable of specifically enhancing the function of clone 901, a physiological ligand and an agonist to this receptor can be mentioned.

Therefore, another aspect of the present invention provides a screening method for a substance having a therapeutic activity against lifestyle-related diseases or cibophobia, which comprises screening for an antagonist or an agonist to clone 901. This method comprises comparing, in a series of receptor—G protein coexpression systems obtained by constructing a constitution unit for a receptor-binding region of each family, wherein one constitution unit comprises a system comprising, as essential elements, at least a lipid bilayer membrane containing clone 901 or an equivalent thereof, and a polypeptide containing at least a receptor-binding region of a G protein α subunit belonging to a certain family (hereinafter to be also referred to as Gα) and a guanine nucleotide-binding region of any G protein α subunit, a GDP/GTP exchange reaction of G protein or the activity of an effector the G protein acts upon, in the presence of a test substance and in the absence of the test substance.

Accordingly, the present invention also provides a screening system for a substance having a therapeutic activity against a lifestyle-related disease or cibophobia, which comprises the above-mentioned series of receptor-G protein coexpression systems.

The present invention moreover provides a therapeutic agent for a lifestyle-related disease or a therapeutic agent for cibophobia, comprising, as an active ingredient, a substance having a therapeutic activity against a lifestyle-related disease or cibophobia, which is obtained by the above-mentioned screening system or screening method.

Further characteristics and advantages of the present invention will become clear from the disclosure of “Best Mode for Embodying the Invention” below.

### BRIEF DESCRIPTION OF THE DRAWINGS

FIG. 1 shows the effect (immediately after administration—12 hours later) of administration of antisense DNA of clone 901 on food intake of normal mouse and obese mouse, wherein black columns show food consumption from immediately after administration to 12 hours later (average±standard deviation, normal mouse n=4, obese mouse n=3) of solvent control solution administration groups, white columns show that of antisense DNA solution administration groups, and * in the Figure indicates the presence of a significant difference between the both groups (p<0.05, Student t-test).

FIG. 2 shows the effect (12 hours after administration—24 hours later) of administration of antisense DNA of clone 901 on food intake of normal mouse and obese mouse, wherein black columns show food consumption from 12 hours after administration to 24 hours later (average±standard deviation, normal mouse n=4, obese mouse n=3) of solvent control solution administration groups and white columns show that of antisense DNA solution administration groups.

FIG. 3 shows cAMP concentrations of HEK293 cell extract in which clone 901 was temporarily expressed alone or on fusing with various Gα proteins, wherein mock shows HEK293 cells transfected with pcDNA3.1Zeo(+), 901 shows HEK293 cells transfected with pc901Zeo, 901-Gq shows HEK293 cells transfected with pc901HISGα16, 901-Gi shows HEK293 cells transfected with pc901HISGαi2 and 901-GS shows HEK293 cells transfected with pc901HISGαS2.

### BEST MODE FOR EMBODYING THE INVENTION

The present invention provides a therapeutic agent for a lifestyle-related disease wherein the active ingredient is a substance that suppresses the expression or functioning of clone 901, a GPCR expressed in the hypothalamus. In general, a lifestyle-related disease is defined as a syndrome wherein lifestyles such as dietary habits, exercise habits, resting, smoking and drinking are involved in the pathogenesis and progression thereof. The same term, as used herein, specifically refers to a “syndrome wherein a therapeutic effect can be achieved by adjusting food intake to reduce it,” typically exemplified by diabetes, obesity, hyperlipidemia, hyperuricemia, etc. Patients often have two or more of these diseases at a time.

“Clone 901” is one of human GPCR proteins consisting of the amino acid sequence shown by SEQ ID NO:2. Although cDNA encoding this receptor has already been isolated from cDNA libraries from fetal liver-spleen (WO 00/00515) or fetal small intestine (WO 00/15793), physiological ligands therefor and conjugating G protein family, etc. remain unknown. The present inventors independently found that this receptor gene is expressed in the human hypothalamus at high levels and conducted further investigations based on this finding. As a result, as described above, the present inventors identified this protein as a membrane receptor involved in feeding center stimulation.

It is well known that GPCRs corresponding to human clone 901 exist in other mammals [e.g., this receptor of mouse origin (registered in GenBank under accession number NM_{—}030258) has 74% homology with human clone 901 at the amino acid level]. Accordingly, the therapeutic agent for a lifestyle-related disease of the present invention is intended for use to treat diabetes, obesity, hyperlipidemia, hyperuricemia, etc., not only in humans but also in other mammals. Because the number of animals suffering from diseases like lifestyle-related diseases such as obesity due to excess feeding and a lack of exercise has been increasing with the recent pet animal boom, the remedy of the present invention is very useful in the field of veterinary medicine as well.

The therapeutic agent for a lifestyle-related disease of the present invention contains as an active ingredient a substance that suppresses the expression or functioning of clone 901. The term expression, as used herein, refers to a state wherein a receptor protein is produced and functionally arranged on the cell membrane. Accordingly, the “substance that suppresses the expression” may act at any stage, such as at the gene transcription level, post-transcription regulation level, translation-into-protein level, post-translational modification level, membrane transport level and protein folding level. On the other hand, the “substance that suppresses the functioning” refers to a substance that acts on a receptor once functionally arranged on the cell membrane, and that does not cause a shift of the equilibration between the active and inactive forms at least toward the active side.

Examples of substances that suppress the expression of clone 901 include transcription suppression factors, RNA polymerase inhibitors, RNA-decomposing enzymes, protein synthesis inhibitors, protein-decomposing enzymes, and protein denaturants; to minimize the adverse effects on other genes and proteins that are expressed in cells, it is important that the substance should be capable of specifically acting on the target molecule. Accordingly, a preferred embodiment of a substance that suppresses the expression of clone 901 is an antisense nucleic acid of the clone 901 mRNA (consisting of the base sequence shown by SEQ ID NO:1) or its initial transcription product. An antisense nucleic acid refers to a nucleic acid that consists of a base sequence capable of hybridizing to target mRNA (initial transcription product) under the physiological conditions of cells that express the target mRNA (initial transcription product), and that is capable of inhibiting the translation of the polypeptide encoded by the target mRNA (initial transcription product) while in the hybridized state. The antisense nucleic acid may be DNA or RNA and may be a DNA/RNA chimera. Additionally, because naturally occurring antisense nucleic acids have their phosphate di-ester linkage decomposed easily by the nucleic acid-decomposing enzyme present in cells, the antisense nucleic acid of the present invention can also be synthesized using modified nucleotides such as of the thiophosphate type (phosphoric acid bond P═O replaced with P═S) or 2′-O-methyl type, which types are stable to the decomposing enzyme. Other important requirements for designing an antisense nucleic acid include increasing the water solubility and cell membrane permeability; these goals can also be achieved by improving the dosage form such as through the use of liposome or microspheres.

The length of the antisense nucleic acid of the present invention is not subject to limitation, as long as the antisense nucleic acid is capable of specifically hybridizing to the clone 901 mRNA or its initial transcription product, and the antisense nucleic acid may be a sequence comprising a sequence of about 15 bases at the shortest in length or complementary to the entire sequence of the mRNA (initial transcription product) at the longest. From the viewpoint of ease of synthesis, antigenicity concern, and other aspects, there may be mentioned, for example, oligonucleotides consisting of preferably about 15 to about 30 bases. When the antisense nucleic acid is an about 25mer oligo DNA, the base sequence capable of hybridizing to the clone 901 mRNA under physiological conditions may be any one, as long as it possesses about 80% homology or more, depending on the base composition of the target sequence.

The target sequence for the antisense nucleic acid of the present invention is not subject to limitation, as long as it is a sequence such that the translation of clone 901 protein or a functional fragment thereof is inhibited as a result of hybridization of the antisense nucleic acid, and may be the entire sequence or a partial sequence of the clone 901 mRNA or may be the intron portion of the initial transcription product. However, when using an oligonucleotide as the antisense nucleic acid, it is desirable that the target sequence should be located between the 5′-terminus of the clone 901 mRNA and the C-terminus of the coding region (the region shown by base numbers 1-1152 in the base sequence shown by SEQ ID NO:1). Preferably, the target sequence is a region on the N-terminus side of the coding region from the 5′-terminus, with greatest preference given to a base sequence in the vicinity of the initiation codon (base number 154). Additionally, it is preferable that the target sequence should be selected such that an antisense nucleic acid complementary thereto does not form a secondary structure such as a hairpin structure.

Furthermore, the antisense nucleic acid of the present invention may be capable of not only hybridizing to the clone 901 mRNA or its initial transcription product to inhibit the translation, but also binding to the clone 901 gene, a double-stranded DNA, to form a triple-strand (triplex) to inhibit the transcription into mRNA.

Another preferred embodiment of a substance that suppresses clone 901 expression is a ribozyme capable of specifically cleaving the clone 901 mRNA or its initial transcription product in the coding region (the base sequence shown by base numbers 154-1152 in the base sequence shown by SEQ ID NO:1) (including the intron portion in case of the initial transcription product). The term ribozyme refers to an RNA possessing an enzyme activity to cleave nucleic acid. Since it has recently been shown that oligo DNA having the base sequence at the enzyme activity site also possesses nucleic acid cleavage activity, the term ribozyme is used herein to include DNA, as long as it possesses sequence-specific nucleic acid cleavage activity. The most widely applicable ribozyme is self-splicing RNA found in infectious RNA of viroids, virusoids, etc.; such ribozymes include the hammerhead type and the hairpin type. The hammerhead type is capable of specifically cleaving the target mRNA alone by exhibiting enzyme activity with about 40 bases, and rendering several bases at each end adjoining to the hammerhead structure (about 10 bases in total) complementary to the desired cleavage-site of the mRNA. This type of ribozymes is also advantageous in that they do not attack genomic DNA because their substrate is RNA alone. When the clone 901 mRNA by itself takes a double-stranded structure, it is possible to render the target sequence single-stranded by using a hybrid ribozyme resulting from the joining of an RNA motif of viral nucleic acid origin that is capable of specifically binding to RNA helicase [Proc. Natl. Acad. Sci. USA, 98(10): 5572-5577 (2001)]. Furthermore, when using the ribozyme in the form of an expression vector containing DNA that encodes the ribozyme, the ribozyme may be a hybrid ribozyme resulting from the further joining of a sequence with tRNA modified to promote the transfer to cytoplasm [Nucleic Acids Res., 29(13): 2780-2788 (2001)].

Another embodiment of a substance that suppresses the expression of clone 901 is a double-stranded oligo RNA that is complementary to a partial sequence in the coding region of the clone 901 mRNA or its initial transcription product (including the intron portion in the case of the initial transcription product). What is called RNA interference (RNAi), a phenomenon wherein upon intracellular introduction of a short double-stranded RNA, an mRNA complementary to that RNA is decomposed, has long been known to occur in nematodes, insects, plants, and other organisms. Since this phenomena has recently been found to occur in animal cells as well [Nature, 411(6836): 494-498 (2001)], RNAi is drawing attention for its potential as an alternative to ribozyme.

The antisense oligonucleotide and ribozyme of the present invention can be prepared by determining the target sequence for the mRNA or its initial transcription product on the basis of the clone 901 cDNA sequence or genomic DNA sequence, and synthesizing a complementary sequence using a commercially available DNA/RNA synthesizer (Applied Biosystems, Beckman, etc.). A double-stranded oligo RNA possessing RNAi activity can be prepared by synthesizing a sense strand and an antisense strand using a DNA/RNA synthesizer, denaturing each strand in the appropriate annealing buffer solution at about 90° C. to about 95° C. for about 1 minute, and subsequently annealing them at about 30° C. to about 70° C. for about 1 to 8 hours. Additionally, a longer double-stranded polynucleotide can be prepared by synthesizing complementary oligonucleotide strands in alternative overlaps, annealing them, and subsequently subjecting them to ligation with ligase.

A preferred embodiment of a substance that suppresses the functional expression of clone 901 at the post-translational level is an antibody against clone 901 or a fragment thereof. This antibody may be a polyclonal antibody or monoclonal antibody, and can be prepared by a well-known immunological technique. Any fragment of the anti-clone 901 antibody serves for the purpose, as long as it has an antigen-binding site (CDR) for clone 901, and is exemplified by Fab, F(ab′)₂, ScFv, minibody, etc.

For example, a polyclonal antibody can be obtained by giving clone 901 protein or a fragment thereof [may be prepared as a complex cross-linked with a carrier protein such as bovine serum albumin or KLH (Keyhole Limpet Hemocyanin), if necessary] as the antigen, along with a commercially available adjuvant (e.g., complete or incomplete Freund's adjuvant), to an animal by subcutaneous or intraperitoneal administration about 2 to 4 times at intervals of 2 to 3 weeks (the antibody titer of serum separated from drawn blood determined by a commonly known antigen-antibody reaction, and its elevation confirmed in advance), collecting whole blood about 3 to about 10 days after final immunization, and purifying the antiserum. Animals to be administered with the antigen include mammals such as rats, mice, rabbits, goat, guinea pigs and hamsters.

A monoclonal antibody can also be prepared by a cell fusion method (e.g., Takeshi Watanabe, saibouyugouhou no genri to monokuronaru kotai no sakusei, Akira Taniuchi and Toshitada Takahashi, eds., “monokuronaru kotai to gan—kiso to rinsho—”, pp. 2-14, Science Forum Publishing, 1985). For example, a mouse is given this factor, along with a commercially available adjuvant, 2 to 4 times by subcutaneous or intraperitoneal administration, its spleen or lymph node is collected about 3 days after final administration, and leukocytes are separated. These leukocytes are fused with myeloma cells (e.g., NS-1, P3X63Ag8, etc.) to yield a hybridoma that produces a monoclonal antibody against this factor. The cell fusion may be achieved by the PEG method [J. Immunol. Methods, 81(2): 223-228 (1985)] or the voltage pulsation method [Hybridoma, 7(6): 627-633 (1988)]. A hybridoma that produces the desired monoclonal antibody can be selected by detecting in the culture supernatant an antibody that specifically binds to an antigen using well-known EIA, RIA, or the like. Cultivation of a hybridoma that produces a monoclonal antibody can be conducted in vitro, or in vivo in mice or rats, preferably in mouse ascites fluid, and the resulting antibody can be obtained from a hybridoma culture supernatant or animal ascites fluid, respectively.

However, in view of therapeutic effect and safety in humans, the anti-clone 901 antibody of the present invention is preferably a chimeric antibody between a human and another animal (e.g., mice etc.), more preferably a humanized antibody. The term chimeric antibody, as used herein, refers to an antibody having a variable region (V region) from an immunized animal and a constant region (C region) from a human; “humanized antibody” refers to an antibody wherein all regions except CDR have been replaced with a human antibody. A chimeric antibody or a humanized antibody can, for example, be obtained by cutting out a sequence that encodes a V region or CDR from the gene for a mouse monoclonal antibody prepared in the same manner as above, cloning a chimeric gene resulting from fusion with DNA that encodes a C region of an antibody from human myeloma into an appropriate expression vector, and introducing the vector to an appropriate host cell to express the chimeric gene.

Another preferred embodiment of a substance that suppresses the functional expression of clone 901 at the post-translational level is an oligonucleotide that specifically binds to clone 901 and inhibits its functional expression, i.e., aptamer. An aptamer for clone 901 can, for example, be obtained by the procedure shown below. First, oligonucleotides (e.g., about 60 bases) are randomly synthesized using a DNA/RNA synthesizer to obtain a pool of oligonucleotides. Next, the desired protein, i.e., an oligonucleotide that binds to clone 901 is separated using an affinity column. The separated oligonucleotide is amplified by PCR and again screened through the aforementioned selection process. By repeating this process in about five cycles or more, an aptamer showing high affinity for clone 901 can be selected.

A therapeutic agent for a lifestyle-related disease wherein the active ingredient is a substance that suppresses the expression of clone 901 is not capable of exhibiting its therapeutic activity unless it is incorporated in cells of the target tissue (i.e., hypothalamus); its active ingredient, nucleic acid or protein molecule, is not easily absorbable in cells and in addition is likely to undergo rapid decomposition in the body. Additionally, because the uptake of these molecules is usually by endocytosis, they are likely to undergo decomposition by lysosome enzyme. Accordingly, it is important to design a drug delivery system (DDS) wherein a substance that suppresses the expression of clone 901 is delivered to hypothalamic cells in a stable state so as to increase cell membrane permeability and to promote drug release from lysosome/endosome. For example, in the case of an oligo nucleic acid molecule such as an antisense oligonucleotide, it is possible to improve the stability to nuclease, intracellular transfer, and release from lysosome/endosome by chemical modifications such as PNA resulting from the replacement of the phosphoric acid and sugar portions with peptide bonds, and oligonucleotides having a morphine backbone in place of a phosphoric acid backbone, as well as nucleic acids with their phosphate linkage or sugar portion (2′ position, 3′ position, etc.) modified as described above; these modified oligo nucleic acids can easily be prepared using a DNA/RNA synthesizer.

On the other hand, cell membrane permeability can also be increased by coupling an accessory group such as poly-L-lysine, avidin, cholesterol or phospholipid to an oligonucleotide or antibody molecule.

Furthermore, it is also possible to prepare an oligonucleotide or antibody molecule as incorporated in a cationic liposome. By incorporating in a liposome, the active ingredient is protected against decomposition by nuclease and protease, and is incorporated in cells by endocytosis with the cationic surface of the liposome membrane binding to negatively charged molecules on the cell surface. Cationic liposome can, for example, be prepared by mixing a cationic lipid, such as DOTMA, DDAB or DMRIE, and DOPE, a neutral lipid capable of membrane fusion. Because nucleic acid and proteins are polyanionic, they easily form complexes when mixed with cationic liposomes. Additionally, it is possible to achieve cell-specific targeting by inserting in the liposome membrane an antibody or ligand for a cell surface molecule that is expressed specifically in hypothalamus cells. For example, the anti-clone 901 antibody itself may be inserted in the liposome membrane.

Additionally, to protect the liposome incorporated by endocytosis against decomposition by lysosome enzyme, it is also preferable to use a pH-sensitive liposome (at acidic pH levels, the membrane becomes unstable and its contents are released from endosomic vesicles to cytoplasm before fusion with lysosome) or a liposome fused with Sendai virus wherein the viral RNA has been completely fragmented by ultraviolet irradiation etc. (the endocytosis pathway avoided by means of the membrane fusion capability of Sendai virus).

The therapeutic agent for a lifestyle-related disease of the present invention designed in a dosage form as described above can be administered orally or parenterally by dissolving or suspending in an appropriate sterile vehicle. Examples of parenteral administration route include, for example, but are not limited to, systemic administrations such as intravenous, intra-arterial, intramuscular, intraperitoneal and intratracheal administrations, and local administration in the vicinity of the hypothalamus. Preferably, there may be mentioned local administration to the lateral ventricle.

The dosage of the therapeutic agent for a lifestyle-related disease of the present invention varies depending on the kind of active ingredient, molecule size, administration route, severity of disease, animal species of administration subject, drug acceptability of administration subject, body weight, age, etc., and normally ranges from about 0.0008 to about 2.5 mg/kg, preferably about 0.008 to about 0.025 mg/kg, based on the amount of active ingredient per day for each adult; such a dose may be administered at a time or in divided portions.

When the substance that suppresses the expression of clone 901 is a nucleic acid molecule like an antisense nucleic acid, ribozyme or aptamer (hereinafter also referred to as effective nucleic acid molecule), the therapeutic agent for a lifestyle-related disease of the present invention may contain as an active ingredient an expression vector that encodes the effective nucleic acid molecule. With regard to the expression vector, the aforementioned oligonucleotide or polynucleotide that encodes an effective nucleic acid molecule must be functionally joined to a promoter capable of exhibiting promoter activity in hypothalamus cells of the recipient mammal or arranged at a position such that the oligonucleotide or polynucleotide is capable of turning to a form functionally joined under particular conditions in hypothalamus cells of the administration subject. Any promoter can be used, as long as it is capable of working in hypothalamus cells of the recipient mammal; such promoters include, for example, viral promoters such as the SV40-derived initial promoter, cytomegalovirus LTR, Rous sarcoma virus LTR, MoMuLV-derived LTR and adenovirus-derived initial promoter, and mammal constitutive protein gene promoters such as the β-actin gene promoter, PGK gene promoter and transferrin gene promoter. The wording “arranged at a position such that the oligonucleotide or polynucleotide is capable of turning to a form functionally joined under particular conditions” means that, for example, the promoter and the oligo(poly)nucleotide that encodes an effective nucleic acid molecule are separated by a spacer sequence sufficiently long to prevent the expression of the effective nucleic acid molecule from the promoter, split by two recombinase recognition sequences arranged in the same direction, the spacer sequence is cleaved out in the presence of a recombinase that specifically recognizes the recognition sequence, and the polynucleotide that encodes the effective nucleic acid molecule is functionally joined to the promoter as described in more detail below.

The expression vector on the present invention contains a transcription termination signal, i.e., a terminator region, preferably downstream of an oligo (poly) nucleotide that encodes the effective nucleic acid molecule. Furthermore, the expression vector in the present invention may further contain selection marker genes for transformant selection (genes that confer resistance to such drugs as tetracycline, ampicillin, kanamycin, hygromycin and phosphinoslysine, genes that complement auxotrophic mutation, etc.). When the expression vector has a spacer sequence sandwiched by a recombinase recognition sequence as described above, the selection marker gene may also be arranged in the spacer sequence.

The vector for the expression vector in the present invention is not subject to limitation; examples of vectors that are suitable for administration to mammals such as humans include viral vectors such as retrovirus, adenovirus, adeno-associated virus, herpes virus, vaccinia virus, pox virus, polio virus, Sindbis virus and Sendai virus. Adenovirus is advantageous in a number of features, including extremely high gene introduction efficiency and the capability of being introduced into non-splitting cells. It should be noted, however, that because the incorporation of the introduced gene into the host chromosome is extremely rare, this gene expression is transient and usually only lasts for about 4 weeks. In view of the persistence of the therapeutic effect, it is also preferable to use an adeno-associated virus, which is of relatively high gene introduction efficiency, which can be introduced to non-splitting cells as well, and which can be incorporated into chromosomes via an inverted terminal repeat sequence (ITR).

Effective nucleic acid molecules such as of antisense nucleic acids and ribozyme are in essence foreign substances; their constitutive and excess expression is highly toxic to the host animal incorporating that gene and may cause adverse reactions. Accordingly, in a preferred embodiment of the present invention, the expression vector is capable of allowing an effective nucleic acid molecule to express time-specifically and/or hypothalamus cell-specifically to avoid the adverse effects of the excess expression of the effective nucleic acid molecule at an unwanted time and/or unwanted site. As a first example of such a vector, there may be mentioned a vector containing an oligo(poly)nucleotide that encodes an effective nucleic acid molecule joined functionally to a promoter derived from a gene specifically expressed in hypothalamus cells of the administration subject. More specifically, there may be mentioned, for example, the native promoter of the clone 901 gene.

As a second example of the time-specific and hypothalamus-specific expression vector of the present invention, there may be mentioned a vector containing an oligo(poly)nucleotide that encodes an effective nucleic acid molecule functionally joined to an inducible promoter subject to trans-control of the expression by an exogenous substance. When using the metallothionein-1 gene promoter, for example, as the inducible promoter, the expression of the effective nucleic acid molecule can be induced hypothalamus-specifically at any time by locally administering an inducer such as a heavy metal, e.g., gold, zinc and cadmium, a steroid, e.g., dexamethasone, an alkylating agent, a chelating agent or a cytokine to the hypothalamus at the desired time.

Another preferred example of the time-specific and hypothalamus-specific expression vector of the present invention is a vector having a structure wherein the promoter and the oligo(poly)nucleotide that encodes an effective nucleic acid molecule are separated by a spacer sequence sufficiently long to prevent the expression of the effective nucleic acid molecule from the promoter, split by two recombinase recognition sequences arranged in the same direction. Solely introducing the vector in hypothalamus cells does not ensure that the promoter directs the transcription of the effective nucleic acid molecule. However, provided that a recombinase that specifically recognizes the recognition sequence is locally administered to the hypothalamus at the desired time, or an expression vector containing a polynucleotide that encodes recombinase is locally administered to express the recombinase in hypothalamus cells, homologous recombination via the recombinase occurs in the recognition sequences; as a result, the spacer sequence is cleaved out and the oligo(poly)nucleotide that encodes the effective nucleic acid molecule is functionally joined to the promoter, resulting in the hypothalamus-specific expression of the effective nucleic acid molecule at the desired time.

It is desirable that the recombinase recognition sequence used in the aforementioned vector should be a heterologous recombinase recognition sequence that is not recognized by endogenous recombinase so as to prevent the recombination by the recombinase present in the recipient. It is desirable, therefore, that the recombinase that exhibits trans-action on the vector should also be a heterologous recombinase. Preferred examples of such combinations of heterologous recombinase and the recombinase recognition sequence include, but are not limited to a combination of Escherichia coli bacteriophage P1-derived Cre recombinase and the lox P sequence, and a combination of yeast-derived Flp recombinase and the frt sequence.

Discovered in a bacteriophage, Cre recombinase is known to work in the specific DNA recombination reaction, not only in prokaryotic cells but also in animal cells and animal viruses, which are eukaryotic cells. When two lox P sequences are present on the same DNA molecule in the same direction, Cre recombinase cleaves out the DNA sequence sandwiched by the sequences to allow them to form a cyclic molecule (cleavage reaction). On the other hand, in cases where two lox P sequences are present on different DNA molecules one of which is cyclic DNA, the cyclic DNA is inserted to the other DNA molecule via the lox P sequence (insertion reaction) [J. Mol.

Biol., 150: 467-486 (1981); J. Biol. Chem., 259: 1509-1514 (1984); Proc. Natl. Acad. Sci. USA, 81: 1026-1029 (1984)]. Example cleavage reactions are reported in animal cells in culture [Nucleic Acids Res., 17: 147-161 (1989); Gene, 181: 207-212 (1996)], animal viruses [Proc. Natl. Acad. Sci. USA, 85: 5166-5170 (1988); J. Virol., 69: 4600-4606 (1995); Nucleic Acids Res., 23: 3816-3821 (1995)], transgenic mice [Proc. Natl. Acad. Sci. USA, 89: 6232-6236 (1992); Proc. Natl. Acad. Sci. USA, 89: 6861-6865 (1992); Cell, 73: 1155-1164 (1993); Science, 265:103-106 (1994)], etc.

As the promoter for the time-specific and hypothalamus-specific expression vector of the present invention, which is based on the interaction of a recombinase/recombinase recognition sequence, there may preferably be used a virus-derived promoter or a mammal constitutive protein gene promoter to ensure the expression at the desired time and site.

Administration of the therapeutic agent for a lifestyle-related disease of the present invention wherein the active ingredient is an expression vector that encodes an effective nucleic acid molecule is conducted by either the ex vivo method, in which nerve cells of the animal to treat are taken out from the body, cultured, then treated to introduce the vector and returned to the body, and the in vivo method, in which the vector is introduced by directly administering it to the recipient's body. In case of the ex vivo method, vector introduction in the target cell can be achieved by the microinjection method, calcium phosphate co-precipitation method, PEG method, electroporation method, etc. In case of the in vivo method, the viral vector is administered in the form of an injection or the like intravenously, intra-arterially, subcutaneously, intracutaneously, intramuscularly, intraperitoneally or the like. Alternatively, administering a vector by intravenous injection etc. may pose a problem with the production of a neutralizing antibody against the viral vector; however, it is possible to mitigate the adverse effects of the presence of the antibody by locally injecting the vector in the vicinity of the hypothalamus, where the target cell is present, e.g., in the lateral ventricle (in situ method).

Additionally, when using a non-viral vector as the expression vector that encodes an effective nucleic acid molecule, introduction of the expression vector can be conducted by using a high molecular carrier such as a poly-L-lysine-nucleic acid complex or placing in liposome as described above with respect to dosage forms of therapeutic drugs wherein the active ingredient is the effective nucleic acid molecule as is. Alternatively, it is also possible to introduce the vector directly to the target cell using the particle gun method.

When recombinase itself is locally administered as the trans-acting substance in the use of a vector based on recombinase/recombinase recognition sequence interaction, recombinase, for example, may be injected to the hypothalamus on dissolving or suspending in an appropriate sterile vehicle (e.g., artificial cerebrospinal fluid etc.). On the other hand, when a recombinase expression vector is locally administered to the hypothalamus as the trans-acting substance, the recombinase expression vector may be any vector, as long as it possesses an expression cassette wherein the recombinase-encoding polynucleotide is functionally joined to a promoter capable of exhibiting promoter activity in hypothalamus cells of the administration subject. When the promoter used is a constitutive promoter, it is desirable that the vector administered to the hypothalamus to prevent the expression of recombinase at unwanted times should be a vector that rarely. undergoes incorporation in the host cell chromosome, e.g., adenovirus. However, when using an adenovirus vector, the transient expression of recombinase persists for about 4 weeks at most; if the treatment is prolonged, a second or third administration will be necessary. As another approach to expressing a recombinase at the desired time, there may be mentioned the use of an inducible promoter like the metallothionein gene promoter. In this case, viral vectors of high integration efficiency such as retrovirus can be used.

When the substance that suppresses the expression of clone 901 is a nucleic acid molecule like an antisense nucleic acid, ribozyme or aptamer, the therapeutic agent for a lifestyle-related disease of the present invention may contain as an active ingredient a host cell containing an expression vector that encodes an effective nucleic acid molecule as described above. As examples of useful host cells, there may be mentioned autologous cells taken out as target cells from the recipient in the aforementioned ex vivo introduction method for an expression vector, nerve cells taken out from allogenic (e.g., stillborn fetuses, brain death patients, etc., in case of humans) or heterologous (non-human mammals such as swine and simian, in case of humans) individuals, or nerve cells obtained by culturing and differentiating such nerve stem cells or ES cells. Because the central nervous system is the organ/tissue where graft rejection is most unlikely, even heterologous cells can be allowed to take using a small amount of immunosuppressant in combination.

In another embodiment, it is possible to transform a resident bacterium in the nasal cavity, throat, oral cavity, intestine, or the like of the administration subject as the host cell, with an expression vector that encodes an effective nucleic acid molecule by a conventional method, and to deliver the thus-obtained transformant to a site of the recipient where the host cell normally occurs. In recent years, a pathway, other than the blood-brain barrier, has been investigated via which a drug is transferred from the nose directly to the cerebrospinal fluid for delivery to the brain; the use of a nasal cavity resident bacterium suffices that objective.

The dosage of the therapeutic agent for a lifestyle-related disease of the present invention, wherein the active ingredient is an expression vector that encodes an effective nucleic acid molecule or a host cell harboring the expression vector, varies depending on the kind of active ingredient, molecule size, promoter activity, administration route, severity of disease, animal species of administration subject, drug acceptability of administration subject, body weight, age, etc., and is preferably a level such that an effective nucleic acid molecule, in an amount equivalent to the appropriate dosage of a therapeutic drug wherein the active ingredient is the effective nucleic acid molecule itself, is expressed in the body of an animal receiving a vector or host cell, and is exemplified by about 2 to about 20 μg/kg, preferably about 5 to about 10 μg/kg based on the amount of vector per day for each adult.

Because clone 901 is a membrane receptor protein that mediates signal transduction for positively regulating food consumption, food intake behavior can be induced by enhancing the expression of this receptor. Accordingly, the present invention also provides a therapeutic agent for cibophobia wherein the active ingredient is a substance that enhances the expression of clone 901.

Examples of substances that enhance the expression of clone 901 include trans-activation factors capable of promoting RNA transcription from the clone 901 gene, factors capable of promoting splicing or mRNA transfer to cytoplasm, factors that suppress mRNA decomposition, factors capable of promoting ribosome binding to mRNA, factors that suppress the decomposition of the clone 901 protein, and factors that promote the transportation of the clone 901 protein to the membrane; as preferred examples of more directly acting specific substances, there may be mentioned the clone 901 protein or equivalent thereof, an expression vector containing a nucleic acid that encodes the clone 901, or a host cell harboring the expression vector.

The “clone 901 protein” mentioned here is a protein consisting of the amino acid sequence shown by SEQ ID NO:2; “an equivalent” refers to a polypeptide consisting of an amino acid sequence resulting from the substitution, deletion, insertion, addition or modification of 1 or more (preferably 1 to 50, more preferably 1 to 30, still more preferably 1 to 10, and most preferably 1 to 5) amino acids in the amino acid sequence shown by SEQ ID NO:2, that exhibits a ligand-receptor interaction equivalent to that of a protein consisting of the amino acid sequence shown by SEQ ID NO:2, and that couples with Gα to promote the GDP-GTP exchange reaction of the subunit.

The clone 901 protein or an equivalent can be isolated from a membrane-containing fraction derived from the hypothalamus tissue of humans or of other mammals such as bovine, swine, simian, mouse or rat by affinity chromatography using the anti-clone 901 antibody. Alternatively, a DNA clone isolated from a cDNA library or genomic library derived from the tissue with the clone 901 cDNA clone as a probe can be cloned into an appropriate expression vector, introduced to the host cell, expressed, and purified from the membrane-containing fraction of the cell culture by affinity chromatography using the anti-clone 901 antibody, His-tag, GST-tag, or the like. The equivalent may partially incorporate a mutation induced by an artificial treatment such as site-directed mutagenesis based on the clone 901 cDNA sequence (the base sequence shown by base numbers 154-1152 in the base sequence shown by SEQ ID NO:1). Conservative amino acid substitution is well known; those skilled in the art can induce a mutation as appropriate in the clone 901 protein, as long as the clone 901 receptor characteristics remain unchanged. However, because the ligand binding domain and preferably the extracellular loop to which an inverse agonist is capable of binding, and the N-terminal strand must be conserved to high extents, it is desirable that a mutation should not be induced in such regions. Additionally, to retain the activity of Gα to promote the GDP-GTP exchange reaction, it is preferable that the Gα activation domain on the third intracellular loop should also be conserved to high extents.

A therapeutic agent for cibophobia wherein the active ingredient is the clone 901 protein or an equivalent can be modified to increase its cell membrane permeability by coupling an accessory group such as poly-L-lysine, avidin, cholesterol or phospholipid component as described above with respect to a therapeutic agent for a lifestyle-related disease wherein the active ingredient is the anti-clone 901 antibody. Alternatively, this therapeutic agent can also be prepared by placing the clone 901 protein or an equivalent in a cationic liposome. Because proteins are poly-anionic, this therapeutic agent easily forms a complex when mixed with a cationic liposome. Additionally, it is also possible to achieve cell-specific targeting by incorporating into the liposome membrane an antibody or ligand for a cell surface molecule specifically expressed in hypothalamus cells. For example, it is also possible to incorporate the anti-clone 901 antibody (preferably an antibody not possessing antagonist activity or inverse agonist activity) to the liposome membrane.

A therapeutic agent for cibophobia wherein the active ingredient is the clone 901 protein or an equivalent can be administered orally or parenterally on dissolving or suspending in an appropriate sterile vehicle. Examples of parenteral administration route include, for example, but are not limited to, systemic administrations such as intravenous, intra-arterial, intramuscular, intraperitoneal and intratracheal administrations, and local administration in the vicinity of the hypothalamus. Preferably, there may be mentioned local administration to the lateral ventricle.

The dosage of the present therapeutic agent for cibophobia varies depending on the administration route, severity of disease, animal species of administration subject, drug acceptability of administration subject, body weight, age, etc., and normally ranges from about 0.0008 to about 2.5 mg/kg, preferably about 0.008 to about 0.025 mg/kg based on the amount of active ingredient per day for each adult; such a dose may be administered at a time or in divided portions.

When the substance that enhances the expression of clone 901 is the clone 901 protein or an equivalent, the therapeutic agent for cibophobia of the present invention may be an expression vector containing a nucleic acid that encodes such a polypeptide, and may be a host cell harboring the expression vector. The expression vector and host cell used here may be identical to those used for the aforementioned lifestyle-related disease remedy. Furthermore, regarding the administration route and dosage for these cibophobia remedies, those exemplified above with respect to lifestyle-related disease remedies can be used preferably.

The present invention also provides a therapeutic agent for a lifestyle-related disease wherein the active ingredient is a substance that suppresses the functioning of clone 901 expressed on the cell membrane of hypothalamus cells, or a therapeutic agent for cibophobia wherein the active ingredient is a substance that promotes such function. These therapeutic drugs can be obtained by screening for substances that exhibit agonist activity, antagonist activity or inverse agonist activity to clone 901. Accordingly, the present invention also provides at a time a screening method for a substance that suppresses or promotes the functioning of clone 901, and a screening system for the same method.

The term agonist activity, as used herein, refers to a property by which the substance in question specifically binds to the clone 901 receptor and causes a shift of the equilibration between the active and inactive forms of clone 901 toward the active side. Accordingly, substances possessing agonist activity include physiological ligands for clone 901, as well as what is called full agonists and partial agonists. The term antagonist activity refers to a property by which the substance in question antagonistically binds to the ligand-binding site of clone 901 but has no or almost no effect on the equilibration between the active and inactive forms. Accordingly, substances possessing antagonist activity are understood to be what is called neutral antagonists, and not to include inverse agonists. The term inverse agonist activity refers to a property by which the substance in question binds to any site of clone 901 and causes a shift of the equilibration between the active and inactive forms of clone 901 toward the inactive side. The simple term ligand, as hereinafter used in the present specification, is understood to include all physiological ligands, agonists, antagonists and inverse agonists.

The screening system of the present invention is a series of receptor-G protein co-expression systems obtained by constructing a constituent unit for the receptor-binding region of each Gα family (i.e., Gαₛ, Gαᵢ, Gα_{q}), which constituent unit consists of a lipid bilayer membrane containing the clone 901 protein or an equivalent, and a polypeptide comprising at least the receptor-binding region of a Gα belonging to a family and the guanine nucleotide-binding region of any Gα, as essential member constituents. The clone 901 protein or an equivalent is identical to that mentioned above as an active ingredient of the aforementioned therapeutic agent for cibophobia. Although the lipid bilayer membrane retaining the clone 901 protein or an equivalent may be of any origin, as long as the receptor protein is allowed to take the essential steric structure on the membrane, it is preferably exemplified by fractions containing the cell membrane of eukaryotic cells such as human, bovine, swine, simian, mouse, rat or other mammal cells, and insect cells, e.g., intact cells, cell homogenates, or cell membrane fractions fractionated from these homogenates by centrifugation etc. However, an artificial lipid bilayer membrane prepared by a conventional method from a solution of various lipids, e.g., phosphatidylcholine, phosphatidylserine, and cholesterol, mixed at an appropriate ratio, preferably a ratio close to abundance ratios in the cell membranes of eukaryotic cells such as mammal cells and insect cells, can also be used preferably in an embodiment of the present invention.

Gα(Gαₛ), belonging to the Gₛ family, promotes the activity of adenylate cyclase as the effector, and is exemplified by Gαₛ₋₁-Gαₛ₋₄ and G_{olf}. Gα(Gαᵢ), belonging to the Gᵢ family, suppresses the activity of adenylate cyclase as the effector, and is exemplified by Gαᵢ-Gαᵢ₋₃ and G_{z}. G_{α(Gα}_{q}) belonging to the G_{q} family, promotes the activity of phospholipase C as the effector, and is exemplified by Gα_{q} and G₁₆. The Gαₛ polypeptide, Gαᵢ polypeptide and Gα_{q} polypeptide of the present screening system may lack a portion thereof, as long as each has a region involved in the binding to its own GPCR (RB region) and a region involved in the binding to any guanine nucleotide of Gα (GB region). Results of X-ray crystallographic analysis of Gα have shown that the RB region is located in the vicinity of the C-terminus whereas the GB region is a region homologous to the nucleotide-binding site of the ras protein (from the N-terminus side: amino acid motives called the P box, G′ box, G box, and G″ box, the leader of the αE helix in a highly helical domain, αF helix, etc.). When a physiological ligand or agonist to clone 901 binds to the receptor, the Gα activation domain of the receptor and the Gα RB region that couples with the receptor interact with each other to produce a conformational change in Gα, resulting in the dissociation of GDP from the GB region and quick binding of GTP. Gα-GTP acts on the effector to promote or suppress its activity. On the other hand, binding an inverse agonist inactivates the Gα active domain due to a conformational change in the receptor, resulting in a decreased active Gα-GTP level and inhibition of its action on the effector. Here, provided that a GTP analogue that does not undergo hydrolysis by the GTPase activity of Gα, such as ³⁵S-labeled GTPγS, has been added to the system in place of GTP, it is possible to evaluate the effects of the test material on the GDP-GTP exchange reaction in Gα by determining and comparing the radioactivity bound to the membrane in the presence and absence of the test material, and to screen for substances that possess ligand activity for clone 901. Hence, provided that the radioactivity has increased in the presence of a test material, the test material can be judged to possess agonist activity to clone 901 and hence therapeutic activity for cibophobia. Conversely, provided that the radioactivity has decreased, the test material can be judged to possess inverse agonist activity to clone 901 and hence therapeutic activity for lifestyle-related diseases.

Once ligands for clone 901 are screened for, a family that couples with the receptor is elucidated; subsequent screening can be conducted using only a system containing a Gα poly-petit-peptide belonging to the family as a member constituent. The results described below of permanent activation of clone 901 using a receptor-Gα fusion protein expression system strongly suggest that the G protein a subunit capable of conjugating with clone 901 may be Gαₛ. Accordingly, the present invention also provides a screening method for ligands for the receptor characterized in that the GDP-GTP exchange reaction of the Gα or the activity of the effector that interacts with the Gα is compared in the presence and absence of the test material in a co-expression system of a G protein α subunit (preferably Gαₛ) capable of conjugating with clone 901 and the receptor.

The activity of a ligand for clone 901 can also be determined with the action of a Gα polypeptide on the effector as an index. In this case, the screening system of the present invention must further comprise as another constituent a lipid bilayer membrane containing the effector, in addition to the clone 901 protein or an equivalent. Additionally, the Gα polypeptide must further comprise a region for interaction with the effector. Because the Gα members belonging to the individual families differ with respect to the kind of effector or the direction of action, it is preferable that all Gα polypeptides share the effector interaction region, rather than each has its own effector interaction region. Accordingly, in the present screening system, at least two kinds of Gα polypeptides are chimeric polypeptides containing the effector interaction region of a Gα belonging to another family. For example, when using phospholipase C as the effector, the Gα_{q} polypeptide may be a native one; however, the Gαₛpolypeptide and Gαᵢ polypeptide must be chimeric polypeptides wherein the effector interaction region has been replaced with that of Gα_{q}. As the simplest example of a chimeric polypeptide containing the effector interaction region of a Gα belonging to another family, there may be mentioned a chimeric polypeptide wherein about 5 amino acids at the C-terminus of a Gα belonging to another family (i.e., RB region) have been replaced with its own C-terminal sequence.

In the present screening system, when three kinds of Gα polypeptides contain an effector interaction region of Gαₛ or Gαᵢ, a lipid bilayer membrane containing adenylate cyclase is used as the effector. On the other hand, when the Gα polypeptide contains the effector interaction region of Gα_{q}, a lipid bilayer membrane containing phospholipase C must be used as the effector.

In a screening system comprising adenylate cyclase (hereinafter also referred to as AC) as the effector, the action of a Gα polypeptide on the effector can be evaluated by directly determining the AC activity. The AC activity can be determined using any commonly known technique; examples of useful methods include, but are not limited to, a method comprising adding GTP to an AC-containing membrane fraction, and determining the amount of cAMP produced by competitive immunoassay using an anti-cAMP antibody in the presence of cAMP labeled with a radioisotope (e.g., ³²P), an enzyme (e.g., alkaline phosphatase, peroxidase, etc.), a fluorescent substance (e.g., FITC, rhodamine, etc.), or the like, and a method comprising adding [α-³²P]ATP to an AC-containing membrane, separating the resulting [³²P]cAMP using an alumina column etc., and subsequently determining the radioactivity thereof. When the Gα polypeptide contains the effector interaction region of Gαₛ, and determining and comparing the AC activity in the presence and absence of a test material, provided that the AC activity has increased in the presence of the test material, the test material can be judged to possess agonist activity to clone 901 and hence therapeutic activity for cibophobia. Conversely, provided that the AC activity has decreased, the test material can be judged to possess inverse agonist activity to clone 901 and hence therapeutic activity for lifestyle-related diseases. On the other hand, when the Gα polypeptide contains the effector interaction region of Gα, provided that the AC activity has increased in the presence of a test material, the test material can be judged to possess inverse agonist activity to clone 901 and hence therapeutic activity for lifestyle-related diseases. Conversely, provided that the AC activity has decreased, the test material can be judged to possess agonist activity to clone 901 and hence therapeutic activity for cibophobia.

When using an intact eukaryotic cell as the screening system, the action of a Gα polypeptide on AC can also be evaluated by determining the intracellular cAMP content. Although the intracellular cAMP content can be determined by incubating cells in the presence and absence of the test material for an appropriate time, subsequently disrupting the cells, and subjecting the thus-obtained extract by the aforementioned competitive immunoassay etc., any other commonly known method can be used.

In another embodiment, the cAMP content may be evaluated by determining the amount of expression of reporter gene under the control of the cAMP-responding element (CRE). The expression vector used here is described in detail below, and is outlined below. The intracellular cAMP content is determined by culturing a eukaryotic cell incorporating a vector containing an expression cassette with a DNA that encodes the reporter protein joined downstream of a CRE-containing promoter, in the presence and absence of the test material for an appropriate time, disrupting the cells, and measuring and comparing the expression of the reporter gene in the thus-obtained extract using a commonly known technique.

Accordingly, when the Gα polypeptide contains the effector interaction region of Gαₛ, provided that the intracellular cAMP content (or the amount of expression of reporter gene under the control of CRE) has increased in the presence of a test material, the test material can be judged to possess agonist activity to clone 901 and hence therapeutic activity for cibophobia. Conversely, provided that the cAMP content (or the amount of expression of reporter gene) has decreased, the test material can be judged to possess inverse agonist activity to clone 901 and hence therapeutic activity for lifestyle-related diseases. On the other hand, when the Gα polypeptide contains the effector interaction region of Gαᵢ, provided that the intracellular cAMP content (or the amount of expression of reporter gene under the control of CRE) has increased in the presence of a test material, the test material can be judged to possess inverse agonist activity to clone 901 and hence therapeutic activity for lifestyle-related diseases. Conversely, provided that the cAMP content (or the amount of expression of reporter gene) has decreased, the test material can be judged to possess agonist activity to clone 901 and hence therapeutic activity for cibophobia.

On the other hand, in a screening system containing phospholipase C (hereinafter also referred to as PLC) as the effector (i.e., a case wherein the Gα polypeptide contains the effector interaction region of Gα_{q}), the action of the Gα polypeptide on the effector can be evaluated by directly determining the PLC activity. The PLC activity can, for example, be evaluated by adding ³H-labeled phosphatidylinositol 4,5-diphosphate to a PLC-containing membrane fraction, and determining the amount of inositol phosphate produced using a commonly known technique. The PLC activity is determined and compared in the presence and absence of the test material; provided that the PLC activity has increased in the presence of the test material, the test material can be judged to possess agonist activity to clone 901 and hence therapeutic activity for cibophobia. Conversely, provided that the PLC activity has decreased, the test material can be judged to possess inverse agonist activity to clone 901 and hence therapeutic activity for lifestyle-related diseases.

When using an intact eukaryotic cell as the screening system, the action of Gα polypeptide on PLC can also be evaluated by adding [³H]inositol to the cell and determining the radioactivity of the resulting [³H]inositol phosphate, or determining the intracellular Ca²⁺ content. Although the intracellular Ca²⁺ content can be determined by spectroscopy using a fluorescent probe (fura-2, indo-1, fluor-3, Calcium-Green I, etc.) or by using a calcium-sensitive luminescent protein such as aequorin after the cells are incubated for a given time in the presence and absence of the test material, any other commonly known method can be used. As an apparatus suitable for spectroscopy using a fluorescent probe, there may be mentioned the FLIPR (Molecular Devices Company) system.

In another embodiment, the Ca²⁺ content may be evaluated by determining the amount of expression of reporter gene under the control of Ca²⁺-upregulated TPA (12-O-tetradecanoylphorbol-13-acetate)-responding element (TRE). The expression vector used in that method is described in detail below, and is outlined below. The intracellular Ca²⁺ content is determined by culturing a eukaryotic cell incorporating a vector containing an expression cassette with a DNA that encodes the reporter protein joined downstream of a TRE-containing promoter, in the presence and absence of the test material for an appropriate time, disrupting the cells, and measuring and comparing the expression of the reporter gene in the thus-obtained extract using a commonly known technique.

Accordingly, provided that the intracellular Ca²⁺ content (or the amount of expression of reporter gene under the control of TRE) has increased in the presence of a test material, the test material can be judged to possess agonist activity to clone 901 and hence therapeutic activity for cibophobia. Conversely, provided that the intracellular Ca²⁺ content (or the amount of expression of reporter gene) has decreased, the test material can be judged to possess inverse agonist activity to clone 901 and hence therapeutic activity for lifestyle-related diseases.

The substance subjected to the screening method of the present invention may be any commonly known compound or a new compound, and is exemplified by compound libraries prepared using combinatorial chemistry techniques, random peptide libraries prepared by solid phase synthesis or the phage display method, and naturally occurring components such as those derived from microorganisms, animals, plants, and marine organisms.

A preferred embodiment of a system containing as essential constituents a lipid bilayer membrane containing the clone 901 protein or an equivalent, and Gα polypeptide, which system is a member unit of the screening system of the present invention, is a host eukaryotic cell transfected with both an expression vector containing a DNA that encodes the clone 901 protein or an equivalent and an expression vector containing a DNA that encodes a polypeptide at least comprising the RB region of a Gα belonging to a family and the GB region of any Gα, a homogenate of the cell, or a membrane fraction from the cell.

The “DNA that encodes the clone 901 protein or an equivalent” is not subject to limitation, as long as it is a DNA that encodes a polypeptide consisting of the amino acid sequence shown by SEQ ID NO:2 or a polypeptide that consists of an amino acid sequence resulting from the substitution, deletion, insertion, addition or modification of 1 or more (preferably 1 to 50, more preferably 1 to 30, still more preferably 1 to 10, and most preferably 1 to 5) amino acids in the amino acid sequence shown by SEQ ID NO:2, that exhibits a ligand-receptor interaction equivalent to that of clone 901, and that couples with Gα to promote the GDP-GTP exchange reaction of the subunit. As such DNAs, there may be mentioned, for example the clone 901 cDNA coding region (the base sequence shown by base numbers 154˜1152 in the base sequence shown by SEQ ID NO:1), as well as DNAs that encodes a GPCR corresponding to clone 901 of non-human mammal origin such as of bovine, swine, simian, mouse, or rat; these can be isolated from cDNA libraries or genomic libraries derived from cerebral nerve tissue, including the mammal hypothalamus using the clone 901 cDNA as a probe. The equivalent may partially incorporate a mutation induced by an artificial treatment such as site-directed mutagenesis based on the clone 901 cDNA.

The DNAs that encode the three kinds of Gα polypeptides may lack a portion of the total coding sequence of Gα, as long as each has at least a sequence that encodes the RB region of the Gα in each family, and a sequence that encodes the GB region of any Gα. The sequences of the various Gα genes are commonly known and the RB region and GB region are well known from the results of X-ray crystallographic analysis of Gα as described above. Accordingly, those skilled in the art can easily construct a fragment lacking a portion of the coding sequence of Gα as desired.

In a screening system based on the action of Gα polypeptide on the effector as an index, the DNA that encodes the Gα polypeptide must further contain a nucleic acid sequence that encodes the effector interaction region. Because the three kinds of Gα polypeptides share the effector interaction region as described above, at least two kinds of Gα polypeptides are chimeras having the effector interaction region of different families. As the simplest example of a DNA that encodes the chimeric polypeptide, there may be mentioned a chimeric polypeptide wherein about 5 amino acids at the C-terminus of a Gα cDNA containing the desired effector interaction region have been replaced with a DNA sequence that encodes the C-terminal sequence of a Gα belonging to another family.

The DNA that encodes the clone 901 protein or an equivalent and the DNA that encodes the Gα polypeptide must be functionally joined to a promoter capable of exhibiting promoter activity in the host eukaryotic cell. Any promoter can be used, as long as it is capable of working in eukaryotic cell; such promoters include, for example, viral promoters such as the SV40-derived initial promoter, cytomegalovirus LTR, Rous sarcoma virus LTR, MoMuLV-derived LTR, adenovirus-derived initial promoter and vaculovirus-derived polyhedrin promoter, and eukaryote-derived constitutive protein gene promoters such as the β-actin gene promoter, PGK gene promoter and transferrin gene promoter. It is preferable that the expression vector used contain in addition to the aforementioned promoter a transcription termination signal, i.e., a terminator region, downstream thereof, and it is desirable that the expression vector has an appropriate restriction endonuclease recognition site, preferably a unique restriction endonuclease recognition site that cleaves the vector only at one position, so that a coding DNA can be inserted between the promoter region and the terminator region. Furthermore, the expression vector may further contain a selection marker gene (drug resistance genes such as for tetracycline, ampicillin, kanamycin, hygromycin and phosphinothricin, auxotrophic mutation complementary genes, etc.).

As examples of vectors useful in the screening system of the present invention, there may be mentioned plasmid vectors, viral vectors that are suitable for use in mammals such as humans, including retrovirus, adenovirus, adeno-associated virus, herpes virus, vaccinia virus, pox virus, polio virus, Sindbis virus and Sendai virus, and vaculovirus vectors that are suitable for use in insect cells.

The DNA that encodes the clone 901 protein or an equivalent and the DNA that encodes the Gα polypeptide may be co-transfected to the host cell as carried on two separate expression vectors, or introduced to the host cell as inserted in a single vector dicistronically or monocistronically.

The host cell may be any one, as long as it is a eukaryotic cell such as a mammal cell such as a human, simian, mouse, rat or hamster cell, or an insect cell. Specifically, such host cells include mouse-derived cells such as COP, L, C127, Sp2/0, NS-1, NIH3T3 and ST2, rat-derived cells, hamster-derived cells such as BHK and CHO, simian-derived cells such as COS1, COS3, COS7, CV1 and Vero, and human-derived cells such as HeLa and 293, as well as insect-derived cells such as Sf9, Sf21 and High Five.

Gene introduction to the host cell can be achieved using any commonly known method applicable to gene introduction to eukaryotic cells; examples of such methods include the calcium phosphate co-precipitation method, the electroporation method, the liposome method, and the microinjection method.

The gene-incorporating host cell can, for example, be cultured using a minimum essential medium (MEM) containing about 5% to about 20% bovine fetal serum, Dulbecco's modified Eagle medium (DMEM), RPMI1640 medium, 199 medium, Grace's insect cell culture medium, etc. Medium pH is preferably about 6 to about 8; culturing temperature is normally about 27° C. to about 40° C.

The thus-obtained eukaryotic cell incorporating a DNA that encodes the clone 901 protein or an equivalent and a DNA that encodes a Gα polypeptide may be used as an intact cell as is, depending on the screening method used, or may be used in the form of a cell homogenate obtained by disrupting the cell in an appropriate buffer solution, or a membrane fraction isolated by centrifuging the homogenate under appropriate conditions (e.g., supernatant recovered via centrifugation at about 10,000×g, followed by centrifugation at about 100,000×g and recovery of the sediment).

For example, when the ligand characteristics of the test material are evaluated by GTPγS-binding assay or direct determination of the effector activity, the screening system used is preferably a membrane fraction prepared from cells as described above. On the other hand, when the ligand characteristics of the test material are evaluated by determining the intracellular cAMP content (or the amount of expression of cAMP-responding reporter) or intracellular Ca²⁺ content (or the amount of expression of Ca²⁺-responding reporter), the screening system used is an intact eukaryotic cell.

When evaluating ligand activity with the amount of expression of a cAMP-responding reporter (in cases where the effector is adenylate cyclase) or Ca²⁺-responding reporter (in cases where the effector is phospholipase C) as an index, the host eukaryotic cell must incorporate a vector containing an expression cassette wherein a DNA that encodes the reporter protein is functionally joined downstream of a promoter region containing a cAMP-responding element (CRE) or TPA-responding element (TRE). CRE is a cis-element that activates gene transcription in the presence of cAMP, exemplified by a sequence containing TGACGTCA as a consensus sequence, and may be a sequence containing a deletion, substitution, insertion or addition, as long as cAMP responsiveness is retained. On the other hand, TRE is a cis-element that activates gene transcription in the presence of Ca²⁺, exemplified by a sequence containing TGACTCA as a consensus sequence, and may be a sequence containing a deletion, substitution, insertion or addition, as long as Ca²⁺ responsiveness is retained. As a CRE- or TRE-containing promoter sequence, there may be used in the same manner virus promoters and eukaryotic animal constitutive protein gene promoters as described above; using a restriction endonuclease and DNA ligase, or by means of PCR etc., the CRE or TRE sequence can be inserted downstream of the promoter sequence. As the reporter gene under the control of CRE or TRE, there may be used any commonly known gene that permits quick and simple detection and quantitation of the expression thereof; such genes include, for example, but are not limited to DNAs that encode such reporter proteins as luciferase, β-galactosidase, β-glucuronidase, alkaline phosphatase and peroxidase. More preferably, a terminator sequence is arranged downstream of the reporter gene. As such a vector carrying a CRE (or TRE)-reporter expression cassette, there may be used a commonly known plasmid vector or viral vector.

Another preferred example of a system containing as essential constituents a lipid bilayer membrane containing the clone 901 protein or an equivalent, and a Gα polypeptide, which system is a member unit of the screening system of the present invention, is a host eukaryotic cell transfected with an expression vector containing a DNA that encodes a fused protein wherein a polypeptide at least comprising the RB region of a Gα belonging to a family and the GB region of any Gα is joined to the C-terminus side of the clone 901 protein or an equivalent, a homogenate of the cell, or a membrane fraction from the cell.

A DNA encoding clone 901 protein or an equivalent thereof, and a DNA encoding a polypeptide containing an RB-binding region of Gα of each family and a GB region of any Gα can be obtained as mentioned above. Those of ordinary skill in the art can easily construct a DNA encoding a fused protein of clone 901 and Gα polypeptide by appropriately combining known genetic engineering methods based on these DNA sequences. For example, a method comprising removing a termination codon of a DNA encoding clone 901 by PCR and the like and ligating DNA which the termination codon has been removed with a DNA encoding Gα polypeptide, using a DNA ligase such that the frames match and the like can be mentioned.

A DNA encoding the obtained fused protein is inserted into an expression vector as mentioned above, and introduced into an eucaryotic host cell by the above-mentioned gene introduction technique. When the fused protein is expressed on the obtained eucaryotic cell membrane, and when clone 901 and Gα can interact, Gα active domain on intracellular loop 3 of the receptor and RB region of Gα interact in the absence of a physiological ligand for the receptor, and can promote the GDP/GTP exchange reaction in Gα. In other words, Gα stays constitutively being activated. In contrast, a fused protein with Gα that does not interact with clone 901 does not activate clone 901, and Gα-GTP level does not increase. Here, when a GTP analog free of hydrolysis by GTPase activity of Gα, such as ³⁵S-labeled GTPγS and the like, is added to the system instead of GTP, activation of clone 901 can be evaluated by measuring the radioactivity bound with the membrane and comparing with each other in the membrane systems respectively containing three kinds of fused proteins, thereby identifying the Gα capable of interaction with the receptor.

Once a Gα capable of interaction with clone 901 is identified, the subsequent screening can be conducted using only a membrane system containing clone 901 and the Gα, preferably only a membrane system containing a fused protein of clone 901 and Gα. In other words, in the same manner as in the identification of the above-mentioned coupled Gα, the effect of a test substance on the GDP/GTP exchange reaction in Gα can be evaluated by adding, to the system, a GTP analog free of hydrolysis due to GTPase activity of Gα, and measuring and comparing the radioactivities bound with the membrane in the presence of a test substance and in the absence of the test substance, and a substance having a GPCR ligand activity can be screened for. When the radioactivity increases in the presence of a test substance, the test substance has an agonist activity to clone 901, and when the radioactivity decreases, the test substance has an inverse agonist activity to clone 901. Since a receptor is activated only partially by a fused protein, when a physiological ligand or an agonist to clone 901 is bound, the activity-non-activity balance of the receptor shifts toward the active side, and the Gα-GTP level increases further. Thus, this screening system can screen for agonists as well.

As shown in the Examples below, because clone 901 is constitutively activated only when expressed as a fused protein with Gαₛ, Gα coupled with the receptor is strongly suggested to be Gαₛ. Therefore, the present invention also provides a screening method for a ligand for the receptor, which comprises comparing, in a fused protein expression system of Gαₛ and the receptor, a GDP/GTP exchange reaction of Gαₛ in the presence of a test substance and in the absence of the test substance.

Activation of clone 901 in a fused protein can be also evaluated using, as an index, an action of Gα on an effector. In this case, the screening system of the present invention needs to be a membrane system encompassing, in addition to each fused protein, a lipid bilayer further containing an effector each Gα interacts with. That is, a membrane system containing a fused protein with Gα_{q} further contains phospholipase C (PLC), a membrane system containing a fused protein with Gαᵢ and Gαₛ further contains adenyl cyclase (AC). In this case, the presence or absence of activation of clone 901 can be also evaluated by preparing, for each Gα, a membrane system containing clone 901 and Gα separately (that is, not as a fused protein), and measuring and comparing the activity of effector (that is, in a membrane system containing a fused protein of clone 901 and Gα capable of interaction, the activity of effector is significantly high (low in the case of Gαᵢ) as compared to a membrane system containing the both as non-fused proteins, and for those that do not interact, there is no significant difference in the activity of effector between the both systems).

Once a Gα capable of interaction with clone 901 is identified, the subsequent screening can be conducted using only a membrane system containing a fused protein with said Gα by directly or indirectly measuring and comparing the activity of an effector the Gα can interact with, in the presence of a test substance and in the absence of the test substance.

Therefore, the present invention also provides a screening method for a ligand for clone 901, which comprises measuring and comparing, in a membrane system containing a fused protein of Gα capable of being coupled with a receptor identified by the above-mentioned identification method of Gα coupled with clone 901 and the receptor and an effector with which said Gα is capable of interaction, the activity of an effector, in the presence of a test substance and in the absence of the test substance.

As mentioned above, because clone 901 is constitutively activated only when expressed as a fused protein with Gαₛ; Gα coupled with the receptor is strongly suggested to be Gαₛ. Therefore, the AC activity in a membrane system containing a fused protein of clone 901 and Gαₛ, and AC is measured and compared in the presence of a test substance and in the absence of the test substance. The AC activity can be measured in the same manner as in the above.

It is also known that Gα can be constitutively activated by introducing a mutation by a known method into a specific part of a DNA that encodes Gα and modifying the amino acid sequence thereof. Accordingly, this system can be used similarly for screening for a ligand. Such technique can be performed according to the method described in, for example, Mol. Pharmacol., 57, 890-898 (2000) and Biochemistry, 37, 8253-8261 (1998).

For such fused protein (or mutant Gα) expression cell, too, any form of intact cell, cell homogenate and membrane fraction can be appropriately selected and used according to the screening method to be employed.

In another embodiment of the present invention, as a screening system containing, as constituent elements, a lipid bilayer membrane containing clone 901 protein or an equivalent thereof, and Gα polypeptide, one obtained by re-constituting purified clone 901 protein or an equivalent thereof with Gα polypeptide, or a purified fused protein of the receptor with Gα, in an artificial lipid bilayer membrane can be used. The clone 901 protein or an equivalent thereof can be purified by affinity chromatography with the use of anti-clone 901 antibody and the like from membrane fraction obtained from cerebral nerve tissue and the like including hypothalamus of human or other mammals. Alternatively, the receptor can be purified by affinity chromatography using anti-clone 901 antibody, His-tag, GST-tag and the like, from a recombination cell into which an expression vector containing a DNA encoding clone 901 protein or an equivalent thereof has been introduced. Similarly, a fused protein of the receptor and Gα can be also purified by affinity chromatography using anti-clone 901 antibody, His-tag, GST-tag and the like, from a recombination cell into which an expression vector containing a DNA encoding the fused protein has been introduced.

As a lipid composing an artificial lipid bilayer membrane, phosphatidyl choline (PC), phosphatidyl serine (PS), cholesterol (Ch), phosphatidyl inositol (PI), phosphatidyl ethanolamine (PE) and the like can be mentioned. A mixture of one or more kinds thereof mixed at a suitable ratio is preferably used.

For example, an artificial lipid bilayer membrane (proteoliposome) incorporating a receptor and Gα or a receptor-Gα fused protein can be prepared by the following methods. First, a suitable amount of a mixed lipid chloroform solution of PC:PS:Ch=12:12:1 is separated in a glass tube, chloroform is evaporated in a nitrogen gas vapor to dry the lipid in the form of a film, a suitable buffer is added to suspend the lipid, which is uniformly dispersed by ultrasonication, a buffer containing a surfactant such as sodium cholate and the like is further added to completely suspend the lipid. Thereto is added a suitable amount of purified receptor and Gα, or a receptor-Gα fused protein, and after incubation for about 20-30 min while sometimes stirring in ice water, dialyzed against a suitable buffer, centrifuged at about 100,000×g for 30-60 min and the sediment is recovered to give a desired proteoliposome.

A substance having a therapeutic activity against a lifestyle-related disease or cibophobia, which is selected by a screening system or a screening method as mentioned above can be prepared into a therapeutic agent for a lifestyle-related disease or cibophobia by combining any pharmaceutically acceptable carrier.

Accordingly, the present invention provides a therapeutic agent for a lifestyle-related disease, which comprises, as an active ingredient, an antagonist or an inverse agonist to clone 901, which is selected by the screening method of the present invention. The present invention also provides a therapeutic agent for cibophobia, which comprises, as an active ingredient, a physiological ligand or agonist to clone 901, which is selected by the screening method of the present invention.

The pharmaceutically acceptable carrier is exemplified by, but not limited to, excipients such as sucrose, starch, mannit, sorbit, lactose, glucose, cellulose, talc, calcium phosphate, calcium carbonate and the like, binders such as cellulose, methylcellulose, hydroxypropylcellulose, polypropylpyrrolidone, gelatine, gum arabic, polyethylene glycol, sucrose, starch and the like, disintegrating agents such as starch, carboxymethyl cellulose, hydroxypropyl starch, sodium-glycol-starch, sodium hydrogen carbonate, calcium phosphate, calcium citrate and the like, lubricants such as magnesium stearate, aerosil, talc, sodium lauryl sulfate and the like, aromatics such as citric acid, menthol, glycyl lysine ammonium salt, glycine, orange powder and the like, preservatives such as sodium benzoate, sodium bisulfite, methylparaben, propylparaben and the like, stabilizers such as citric acid, sodium citrate, acetic acid and the like, suspending agents such as methylcellulose, polyvinylpyrrolidone, aluminum stearate and the like, dispersing agents such as surfactant and the like, diluents such as water, physiological saline, orange juice and the like, base wax such as cacao butter, polyethylene glycol, refined kerosene and the like, and the like.

A preparation which is suitable for oral administration is, for example, a liquid comprising an effective amount of a ligand dissolved in a diluent such as water, physiological saline and orange juice, a capsule, sachet or tablet comprising an effective amount of a ligand as a solid or granules, a suspension comprising an effective amount of a ligand in a suitable dispersion medium, an emulsion comprising a solution of an effective amount of a ligand dispersed and emulsified in a suitable dispersion medium and the like.

A preparation preferable for parenteral administration (e.g., subcutaneous injection, intramuscular injection, topical injection, intraperitoneal administration and the like) includes, for example, an aqueous or non-aqueous isotonic sterile injection which may contain antioxidant, buffer, bacteriostatic agent, isotonicity agent and the like. It may be an aqueous or non-aqueous sterile suspension which may contain suspension, solubilizer, thickener, stabilizer, preservative and the like. When the administration method is topical injection near the hypothalamus, an injection containing ligand as an active ingredient dissolved or suspended in an artificial cerebrospinal fluid is preferable. Alternatively, it can be formulated into a sustained release preparation using a biocompatible material such as collagen and the like. Since pluronic gel gelates at body temperature and becomes a liquid at a lower temperature, long duration can be afforded by topically injecting the ligand along with pluronic gel to allow for gelation around the target tissue. The ligand preparation can be sealed in a container in a unit dose or plural doses like an ampoule or vial. It is also possible to lyophilize a ligand and a pharmaceutically acceptable carrier and preserve them in a state that only requires dissolving or suspending in a suitable sterile vehicle immediately before use.

While the dose of the ligand preparation of the present invention varies depending on ligand activity (full agonist or partial agonist, or an antagonist or inverse agonist), degree of seriousness of the disease, the animal species to be the administration subject, drug acceptability, body weight and age of the administration subject, and the like, it is generally about 0.0008-about 2.5 mg/kg, preferably about 0.008-about 0.025 mg/kg, a day for an adult in the amount of the ligand.

The present invention is explained in more detail by referring to Examples, which are mere exemplification and not to be construed as limitative. Unless particularly specified, the following examples were performed according to the methods described in Sambrook, J. et al., Molecular Cloning: A Laboratory Manual, 2nd ed., Cold Spring Harbor Laboratory Press, New York (1989), Current Protocols in Protein Science (ed. Coligan, J. E. et al.), John Wiley and Sons, Inc. and the like.

### EXAMPLE 1

Action of Clone 901 Antisense DNA in Normal and Obesity Model Mice

(1) Test Materials

reagent: clone 901 antisense DNA was a mixture of two kinds of 25mer antisense DNAs relative to the vicinity of the gene initiation codon and synthesis, thiolation and HPLC purification were committed to Nihon Bio Service Co., Ltd.

The base sequences of the antisense DNAs are shown in the following.

| | |
|---|---|
| 5′-GCTTCGCAGCGCTCGCTGGGCGGCG-3′ | (SEQ ID; No 3) |
| 5′-AGTGGGCCACAGCTCCACATGGCAG-3′ | (SEQ ID; No 4) |

For other reagents, commercially available reagent chemicals were used.

Test animal: male C57BL/6N (hereinafter normal mouse) and C57BL db/db (hereinafter obese mouse) (SPF grade) were purchased from Charles River, Japan, Inc. and CLEA JAPAN, INC., respectively, and after preliminary breeding, normal mice were used for the test at the age of 9 weeks and the obese mice were used for the test at the age of 11 weeks.

Breeding environment: The mice were bred in a room controlled to a temperature of not lower than 20° C. and not higher than 26° C., relative humidity of not less than 30% and not more than 70%, lighting cycle of 8:00-20:00 lighting and 20:00-8:00 lights-out. During breeding, the mice were allowed a free access to a solid feed (CE-2, CLEA JAPAN) and sterile distilled water.

(2) Preparation of Administration Liquid

As an antisense DNA administration liquid, an artificial cerebrospinal fluid (0.166 g/L CaCl₂, 7.014 g/L NaCl, 0.298 g/L KCl, 0.203 g/L MgCl₂.6H₂O, 2.10 g/L NaHCO₃) containing 7.5 μg/μl of antisense DNA was prepared. In addition, an artificial cerebrospinal fluid free of antisense DNA was used as a solvent control liquid.

(3) Administration of Antisense DNA in Cerebral Ventricle

The normal mice and obese mice were divided into two groups each, and after fasting overnight, an antisense DNA was administered at 4 μl/mouse (30 μg/mouse in the amount of antisense DNA) to one group and a solvent control liquid (4 μl/mouse) was administered into the lateral ventricle of the other group, simultaneously with the lighting at 8:00 a.m.

(4) Effect of Antisense DNA Administration on Food Consumption of Mice

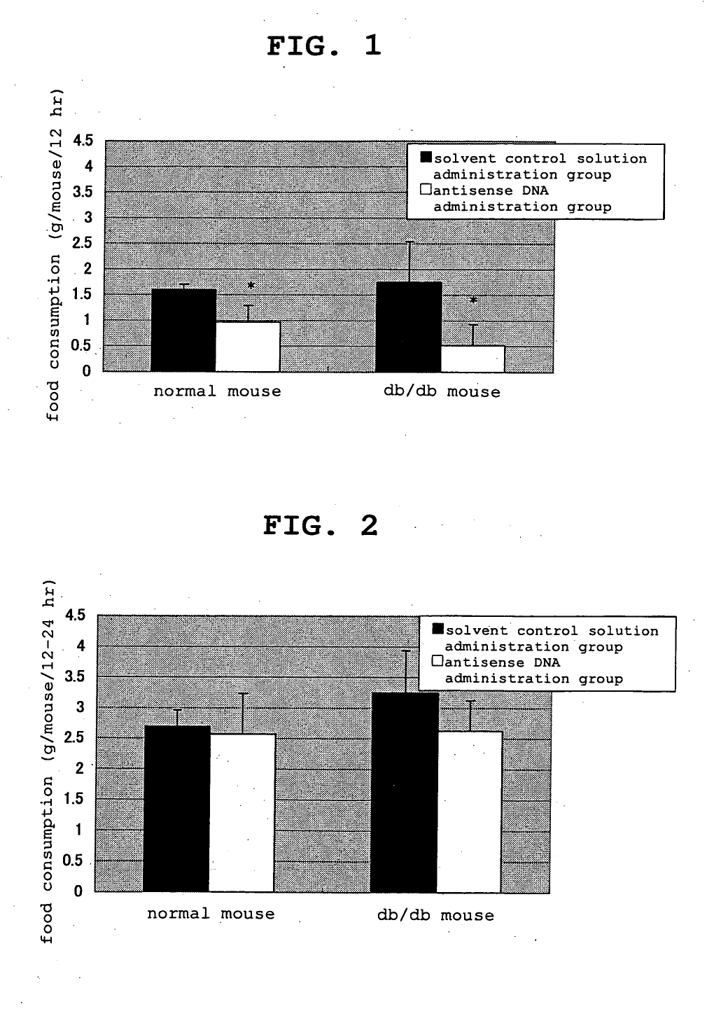

Feeding of mice was resumed immediately after administration, and food consumption was calculated at 12 hours after administration. The effect of the antisense DNA on food consumption of normal mice and obese mice is shown in FIG. 1. By the administration of antisense DNA of clone 901, a food consumption-decreasing effect was found both in normal mice and obese mice. The food consumption from 12 hr to 24 hr after administration was calculated. The effect of the antisense DNA on food consumption of normal mice and obese mice is shown in FIG. 2. The food consumption showed recovery from the effect of administration in 12 hr to 24 hr after administration. In the solvent control liquid administration group, promoted food consumption was found in obese mice as compared to normal mice, and the food consumption is suppressed to the same level with the normal mice by the administration of antisense DNA of clone 901. In the normal mice, no effect of antisense DNA of clone 901 was found.

From the above-mentioned results, the possibility was suggested that clone 901 is a factor involved in feeding behavior. In the obese mice, moreover, the possibility was suggested that the activity or expression amount of clone 901 has been accelerated, wherein an expression suppressive effect was stronger and observed for a longer time due to the antisense DNA. Clone 901 is involved in the control of food consumption in normal animal and overeating obese diabetic animal, and it has been clarified that an effective treatment effect can be afforded by blocking the action of clone 901 particularly when overeating and obesity accompany.

### EXAMPLE 2

Establishment of Clone 901 Stable Expression Cell Line

(1) Construction of Gene-introduced Vector

The coding region of clone 901 is amplified by PCR using KOD-Plus (TOYOBO). A fused gene of the amplified gene fragment and the following three types of chimera Gαs (Gα₁₆, Gα_{qi5}, Gα_{qs5}) (Molecular Devices) are prepared. This fragment is introduced into pcDNA3.1 (Invitrogen).

(2) Establishment of Cell Line

CHO cells are inoculated into a 10 cm² petri dish and cultured in a D-MEM medium (Gibco) containing 10% FBS (Gibco) until 60-70% confluent. The cells are transferred to a serum-free medium. A complex of the introduced gene constructed as mentioned above and Lipoofedtamine-Plus (Gibco) is formed and added to the medium. After incubation for 5 hours, the medium is changed to D-MEM culture medium containing 10% FBS and the cells are further cultured for 8 hours. Then cells are stripped from the petri dish with trypsin-EDTA, suspended in D-MEM medium containing 500 μg/ml of G418 and 10% FBS and inoculated into a 10 cm² petri dish. Colonies formed in several days are isolated and used as clone 901 stable expression cell lines (¥16, ¥qi5, ¥qs5).

### EXAMPLE 3

Screening for Receptor Agonist

(1) Preparation of Cells

Clone 901 stable expression cell lines (¥16, ¥qi5, ¥qs5) are inoculated into a 96 well culture plate and cultured in a D-MEM medium (Gibco) containing 10% FBS (Gibco) until 60-70% confluent. They are used as expression cell.

(2) Addition of Drug

The medium for cell expression is changed to serum free D-MEM medium one day before use. On evaluation day, each compound (2.5 mM DMSO solution) is diluted with D-MEM medium to an objective concentration. Culture medium in the petri dish is removed, and diluted subject compound, 4 μM Fluo3AM (Teflab.) and 2.5 mM probenecid are added and the mixture is cultured at 37° C. for 60 min. A sample treated in the same manner except that the test compound is not added is prepared for comparison.

(3) Measurement of Concentration of Intracellular Calcium by FLIPR Method

The cells treated as mentioned above are washed with ice-cooled PBS, and suspended in Thyrode's medium (containing 2.5 mM probenecid, 1% gelatin). Absorbance of petri dish at 488 nM, 540 nM is quantitatively determined by FLIPR (Molecular Devices).

### Example 4

Construction of Plasmid for Expression of Clone 901-Gα Fused Protein

As three kinds of representative α subunits considered to almost cover all GPCR signaling, Gα16 was selected from the Gq family, Gαi2 was selected from the Gi family and GαS2 was selected from the Gs family. All these coding regions were PCR cloned into expression vector pcDNA3.1(+). A restriction enzyme EcoRV cleavage site and a sequence containing 6×His tags were added just before each Gα coding region by PCR, whereby plasmids pcHISGα16, pcHISGαi2 and pcHISGαS2 capable of easy fusion with GPCR gene on the 5′ side of each Gα protein were prepared.

That is, human spleen cDNA (Clontech) was diluted 20 times, 1 μl thereof was used as a template for amplification. As the enzyme, employed was KODplus (TOYOBO). However, the Mg²+concentration was 1 mM. For amplification reaction, 20 μl of a reaction solution was used. A reaction buffer attached to KODplus was used. To amplify Gα16 cDNA, primer GNA15F1 (SEQ ID; No 5) and GNA15R1 (SEQ ID; No 6) were used; to amplify Gαi2 cDNA, primer GNAi2F1 (SEQ ID; No 7) and GNAi2R1 (SEQ ID; No 8) were used; and to amplify GαS2 cDNA, primer GS2F1 (SEQ ID; No 9) and GS2R1 (SEQ ID; No 10) were used. For amplification, GeneAmp PCR System 9600 (Applied Biosystems) was used and amplification was conducted under the conditions of 94° C., 2 min→(94° C., 15 sec→68° C., 120 sec)×40 cycles to give a PCR product having an object size. The terminal of each PCR product was phosphorylated, separated and purified by 0.8% agarose gel electrophoresis, cleaved with restriction enzyme EcoRV, ligated with plasmid pcDNA3.1(+) (Invitrogen) treated with CIAP and used to transform Escherichia coli DH5α. Plasmids were purified from the transformant line, and upon confirmation that the restriction enzyme digestion pattern and inserted base sequence were objective ones, the obtained G protein expression plasmids were named pcGα16, pcGαi2 and pcGαS2, respectively.

To fuse clone 901 via HIS tag on N terminal of each G protein, a tag sequence was ligated immediately before initiation codon ATG of each G protein, and a restriction enzyme EcoRV recognition site was further added. That is, PCR was conducted using 10 ng each of pcGα16, pcGαi2 and pcGαS2 as a template, a primer (GNA15ATG:SEQ ID; No 11, GNAi2ATG:SEQ ID; No 12 or GS2ATG:SEQ ID; No 13) and a primer (pcDNARV:SEQ ID; No 14) of vector. Amplification was conducted under the conditions of 94° C., 2 min→(94° C., 15 sec→58° C., 30 sec→68° C., 60 sec)×20 cycles to give a PCR product having the object size. Then HIS2 linker (SEQ ID; No 15) and HIS2 linker(R) (SEQ ID; No 16) were annealed, and after phosphorylation of the terminal, ligated with each PCR product. These were digested with restriction enzymes EcoRV, XhoI, and the object DNA fragments were separated and purified by 0.8% agarose gel electrophoresis. The recovered DNA fragments were ligated with expression plasmid pcDNA3.1(+) digested with EcoRV and XhoI, and used to transform Escherichia coli DH5α. Plasmids were purified from transformant line, and upon confirmation that the restriction enzyme digestion pattern and inserted base sequence were object ones, the obtained plasmids were named pcHISGα16, pcHISGαi2 and pcHISGαS2, respectively.

### Example 5

Construction of Clone 901-Gα Fused Protein Expression Plasmid

A plasmid used to express a fused protein of clone 901 and each Gα protein was constructed by the following method. That is, a clone 901 gene was inserted into a plasmid constructed in Example 4 for expression of each GPCR-Gα fused protein to allow for expression of clone 901 as a fused protein with Gα16, Gαi2 or GαS2. Simultaneously, a plasmid to express clone 901 alone was prepared. First, PCR was conducted using clone 901 gene (SEQ ID; No 1)-containing plasmid (2 ng) as a template, a 5′ side primer (901FATG:SEQ ID; No 17) of clone 901 gene and a 3′ side primer (901RT:SEQ ID; No 18) to amplify immediately before the termination codon or a 3′ side primer (901R0:SEQ ID; No 19) to amplify inclusive of the termination codon. Amplification was conducted using KODplus under the conditions of Mg concentration of 1.2 mM, 94° C., 2 min→(94° C., 15 sec→68° C., 120 sec)×20 cycles to give a PCR product having the object size. The terminal of each PCR product was phosphorylated and separated and purified by 0.8% agarose gel electrophoresis. Three kinds of plasmids pcHISGα16, pcHISGαi2 and pcHISGαS2 for expression of GPCR-Gα fused protein and plasmid pcDNA3.1Zeo(+) for single expression were respectively cleaved with restriction enzyme EcoRV and treated with CIAP. The three kinds of plasmids for expression of GPCR-Gα fused protein were ligated with a PCR product amplified to encode immediately before termination codon of clone 901 and used to transform Escherichia coli DH5α. The plasmid for single expression was ligated with a PCR product amplified to include termination codon of clone 901 and used to transform Escherichia coli DH5α. Plasmids were purified from transformant lines, and upon confirmation that the restriction enzyme digestion pattern and inserted base sequence were object ones, the three kinds of clone 901-Gα fused protein expression plasmids were named pc901HISGα16, pc901HISGαi2 and pc901HISGαS2, respectively. In addition, clone 901 single expression plasmid was named pc901Zeo. The cDNA sequences inserted into pc901HISGα16, pc901HISGαi2 and pc901HISGαS2 are shown in SEQ ID; No 20, SEQ ID; No 22 and SEQ ID; No 24, respectively.

### Example 6

Confirmation of Constitutive Activation by Expression of Clone 901-Gα Fused Protein

The constitutive activation by expression of a fused protein of orphan GPCR with Gα can be confirmed by, when the Gα protein to be coupled upon activation of orphan GPCR is Gs, observation of increase of cAMP. When it is Gq, the activation can be confirmed by observation of production of inositol 3 phosphoric acid (IP3). When it is Gi, it can be confirmed by, for example, observation of decrease of production amount of cAMP activated with forskolin, or observation of production of IP3. An increase in the GTP-binding activity of coupled Gα protein under activation of orphan GPCR can be observed in any Gα protein. Therefore, by evaluating the cell lines, into which each orphan GPCR-Gα fused protein expression plasmid has been temporarily introduced, as regards these parameters, a Gα protein capable of constitutive activation, or a Gα protein the orphan GPCR is originally coupled with, can be predicted. At the same time, a screening method targeting the orphan GPCR is established.

In this Example, with the aim of investigating the combination of clone 901 and a constitutively activated Gα protein, plasmids for expression of fused proteins of clone 901 and three kinds of G proteins (Gα16, Gαi2, GαS2) prepared in Example 5 were temporarily introduced into the HEK293 cell, and the concentration of cAMP in the cell extract was quantitatively determined. To be specific, HEK293 cells were sown at a cell density of 2×10⁴ cells/well (100 μl) in a 96-well PDL coated clear plate (Becton Dickinson) and cultured for 24 hr (antibiotics minus). Plasmid DNA (50 ng) of each of the clone 901-Gα fused protein expression plasmids (pc901HISGα16, pc901HISGαi2 and pc901HISGαS2), clone 901 single expression plasmid (pc901Zeo) or expression vector pcDNA3.1Zeo(+) was diluted with 5 μl of OPTI-MEM medium (Invitrogen) and 0.3 μl of LipofectAmine (Invitrogen) was diluted with 5 μl of OPTI-MEM. Both were mixed and incubated at room temperature for 30 min. Serum-free DMEM medium (40 μl) was added and the mixture was added in a well washed once with serum-free DMEM medium. After culture in a CO₂ incubator for 4 hr, it was washed with DMEM medium supplemented with 10% FBS, transferred to DMEM medium supplemented with 10% FBS and cultured in a CO₂ incubator for 24 hr.

After culture for 24 hr, cAMP was quantitatively determined. For the quantitative determination of cAMP, Cyclic AMP kit of CIS Bio International was used. The cells were washed with PBS(−) and transferred to Hanks Hepes solution containing 50 μl of 0.5 mM IBMX. After 15 min, the Hanks Hepes solution was removed by suction and 40 μl of 0.5% Triton X-100 was added to dissolve cells, whereby a cell extract was obtained. From the extract, 24 μl was placed in a 384-well black plate. cAMP-XL665 (12 μl) was added and the mixture was stirred, then 12 μl of anti cAMP-Cryptate was further added and the mixture was stirred and allowed to react at 4° C. for 1 hr. cAMP was quantitatively determined by TR-FRET method using a multi plate reader ARVO (Wallac). Cryptate was excited at 340 nm and 50 μs later, the fluorescence of XL-665 was simultaneously measured at 665 nm and the fluorescence of Cryptate was simultaneously measured at 615 nm. By FRET, fluorescence at 665 nm is observed only when Cryptate and XL-665 approach to each other. The cAMP in the sample decreases fluorescence at 665 nm by binding with anti cAMP-Cryptate competitively with cAMP-XL665.

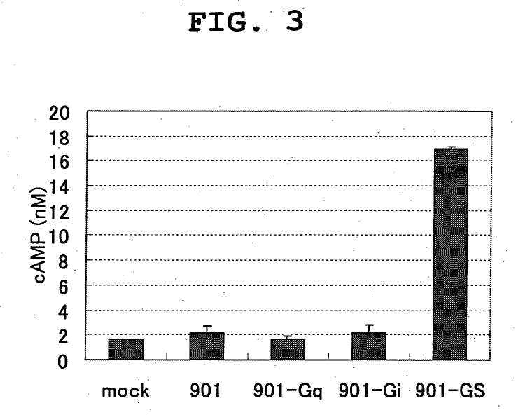

The quantitative determination of cAMP concentration of each cell extract is shown in FIG. 3. A cell extract, into which a plasmid that expresses clone 901 protein (901:pc901Zeo), a fused protein with Gα16 (901-Gq:pc901HISGα16), or a fused protein with Gαi2 (901-Gi:pc901HISGαi2) had been introduced, showed a cAMP concentration of about 2 nM, as in the case of introduction of an expression vector alone (mock:pcDNA3.1Zeo(+)). However, a cell extract, into which a plasmid that expresses a fused protein with GαS2 (901-GS:pc910HISGαS2) had been introduced, showed an about 8.5 times higher cAMP concentration of about 17 nM. That is, constitutive activation was observed only when it fused with GαS protein. The clone 901 was strongly suggested to be a GPCR that coupled with GαS.

Sequence Listing Free Text

SEQ ID; No 3: Oligonucleotide designed to function as an antisense DNA inhibiting expression of clone 901.

SEQ ID; No 4: Oligonucleotide designed to function as an antisense DNA inhibiting expression of clone 901.

SEQ ID; No 5: Oligonucleotide designed to function as a sense primer to amplify human G protein Gα16 cDNA fragment containing full length ORF.

SEQ ID; No 6: Oligonucleotide designed to function as an anti-sense primer to amplify human G protein Gα16 cDNA fragment containing full length ORF.

SEQ ID; No 7: Oligonucleotide designed to function as a sense primer to amplify human G protein Gαi2 cDNA fragment containing full length ORF.

SEQ ID; No 8: Oligonucleotide designed to function as an anti-sense primer to amplify human G protein Gαi2 cDNA fragment containing full length ORF.

SEQ ID; No 9: Oligonucleotide designed to function as a sense primer to amplify human G protein GαS2 cDNA fragment containing full length ORF.

SEQ ID; No 10: Oligonucleotide designed to function as an anti-sense primer to amplify human G protein GαS2 cDNA fragment containing full length ORF.

SEQ ID; No 11: Oligonucleotide designed to function as a sense primer to amplify human G protein Gα16 cDNA fragment from the initiation codon.

SEQ ID; No 12: Oligonucleotide designed to function as a sense primer to amplify human G protein Gαi2 cDNA fragment from the initiation codon.

SEQ ID; No 13: Oligonucleotide designed to function as a sense primer to amplify human G protein GαS2 cDNA fragment from the initiation codon.

SEQ ID; No 14: Oligonucleotide designed to function as an anti-sense primer to amplify multicloning sites of plasmid pcDNA3.1(+)

SEQ ID; No 15: Sense chain oligonucleotide designed to construct a linker containing a nucleotide sequence encoding a 6×His tag peptide sequence.

SEQ ID; No 16: Anti-sense chain oligonucleotide designed to construct a linker containing a nucleotide sequence encoding a 6×His tag peptide sequence.

SEQ ID; No 17: Oligonucleotide designed to function as a sense primer to amplify mRNA of human clone 901 containing full length ORF.

SEQ ID; No 18: Oligonucleotide designed to function as an anti-sense primer to amplify mRNA of human clone 901 up to immediately before termination codon.

SEQ ID; No 19: Oligonucleotide designed to function as an anti-sense primer to amplify mRNA of human clone 901 containing full length ORF.

SEQ ID; No 20: Insert cDNA sequence contained in pc901HISGα16.

SEQ ID; No 22: Insert cDNA sequence contained in pc901HISGαi2.

SEQ ID; No 24: Insert cDNA sequence contained in pc901HISGαS2.

### Industrial Applicability

Since clone 901 is a GPCR involved in feeding behavior, the pharmaceutical composition of the present invention containing, as an active ingredient, a substance that suppresses or enhances expression or function of clone 901 can adjust food intake to a desired level and is expected to afford a therapeutic effect in lifestyle-related diseases caused by overeating, such as diabetes, obesity, hyperlipidemia and the like, or cibophobia. According to the screening system and screening method of the present invention, moreover, a ligand for clone 901 can be easily and rapidly screened for and they are useful for the development of a new drug targeting clone 901, search for a disease marker and establishment of a diagnostic method using the disease marker.

While the present invention has been described with an emphasis on preferred embodiments, it will be obvious to those of ordinary skill in the art that variations of the preferred embodiments may be used. It is intended that the invention may be practiced otherwise than as specifically described herein. Accordingly, this invention includes all modifications encompassed within the spirit and scope of the invention as defined by the following claims.

This application is based on a patent application No. 306872/2001 filed in Japan, the contents of which are hereby incorporated by reference. The references cited herein, including patents and patent applications, are hereby incorporated in their entireties by reference, to the extent that they have been disclosed herein.

1. A therapeutic agent for a lifestyle-related disease comprising, as an active ingredient, a substance that suppresses expression or function of a polypeptide consisting of an amino acid sequence shown in SEQ ID NO:2.

2. A therapeutic agent for a lifestyle-related disease comprising, as an active ingredient, a nucleic acid of the following:
(a) a nucleic acid consisting of a base sequence complementary to a base sequence shown in SEQ ID NO:1, or
(b) a nucleic acid consisting of a base sequence capable of hybridizing with a nucleic acid consisting of a base sequence shown in SEQ ID NO:1 or a primary transcript which generates said base sequence after post-transcriptional processing under physiological conditions of hypothalamus of a subject animal for treatment, and which is capable of inhibiting translation into a polypeptide encoded by the base sequence shown in SEQ ID NO:1 under a hybridized state.

3. A therapeutic agent for a lifestyle-related disease comprising, as an active ingredient, a substance which shows a specific affinity for a polypeptide consisting of an amino acid sequence shown in SEQ ID NO:2 and which inhibits a functional expression of said polypeptide.

4. The therapeutic agent of claim 3, wherein the substance is a nucleic acid.

5. The therapeutic agent of claim 3, wherein the substance is an antibody.

6. A therapeutic agent for a lifestyle-related disease comprising, as an active ingredient, an expression vector encoding the nucleic acid of claim 2.

7. A therapeutic agent for a lifestyle-related disease comprising, as an active ingredient, a host cell transfected with the expression vector of claim 6.

8. The therapeutic agent of any of claims 1 to 7, which is a feeding suppressant, an anti-obesity agent, an anti-diabetic agent or an anti-hyperlipidemic agent.

9. A therapeutic agent for cibophobia comprising, as an active ingredient, a substance that enhances expression or function of a polypeptide consisting of an amino acid sequence shown in SEQ ID NO:2.

10. A therapeutic agent for cibophobia comprising, as an active ingredient, a polypeptide of the following:
(a) a polypeptide consisting of an amino acid sequence shown in SEQ ID NO:2, or
(b) a polypeptide consisting of an amino acid sequence shown in SEQ ID NO:2, wherein one or more amino acids of the amino acid sequence have been substituted, deleted, inserted, added or modified, which shows a ligand—receptor interaction of the same level as the polypeptide of (a), and which is coupled with a G protein α subunit and shows an activity to promote a GDP/GTP exchange reaction of the subunit.

11. A therapeutic agent for cibophobia, comprising, as an active ingredient, an expression vector comprising a nucleic acid encoding the polypeptide of claim 10.

12. A therapeutic agent for cibophobia comprising, as an active ingredient, a host cell transfected with the expression vector of claim 11.

13. A screening system for a substance having a therapeutic activity against a lifestyle-related disease or cibophobia, which comprises, as one constitution unit, a system comprising, as constituent elements, a lipid bilayer membrane comprising a polypeptide of the following:
(a) a polypeptide consisting of an amino acid sequence shown in SEQ ID NO:2, or
(b) a polypeptide consisting of an amino acid sequence shown in SEQ ID NO:2, wherein one or more amino acids of the amino acid sequence have been substituted, deleted, inserted, added or modified, which shows a ligand—receptor interaction of the same level as the polypeptide of (a), and which is coupled with a G protein α subunit and shows an activity to promote a GDP/GTP exchange reaction of the subunit, and a polypeptide comprising at least a receptor-binding region of a G protein α subunit belonging to a certain family and a guanine nucleotide-binding region of any G protein α subunit, wherein said constitution unit is present in a receptor-binding region of each family of the G protein α subunit.

14. The screening system of claim 13, wherein the constitution unit comprises an eucaryotic host cell transfected with an expression vector comprising a DNA encoding the polypeptide of (a) or (b), and an expression vector comprising a DNA encoding a polypeptide comprising at least a receptor-binding region of a G protein α subunit belonging to a certain family and a guanine nucleotide-binding region of any G protein α subunit, a homogenate of said cell or a membrane fraction derived from said cell.

15. The screening system of claim 13, wherein the constitution unit comprises an eucaryotic host cell transfected with an expression vector comprising a DNA encoding a polypeptide obtained by fusion of a polypeptide comprising, on a C terminal of the polypeptide of (a) or (b), at least a receptor-binding region of a G protein α subunit belonging to a certain family and a guanine nucleotide-binding region of any G protein a subunit, a homogenate of said cell or a membrane fraction derived from said cell.

16. The screening system of claim 13, wherein the polypeptide in each constitution unit, which comprises a receptor-binding region of a G protein α subunit and a guanine nucleotide-binding region of any G protein α subunit, further comprises the same effector interaction region and the lipid bilayer membrane further comprises an effector that interacts with said region.

17. The screening system of any of claims 13 to 16, wherein the therapeutic activity against lifestyle-related diseases is a feeding suppressive activity, an anti-obesity activity, an anti-diabetic activity or an anti-hyperlipidemic activity.

18. A screening method for a substance having a therapeutic activity against a lifestyle-related disease or cibophobia, which comprises adding, in each constitution unit of the screening system of claim 13, a labeled GTP analog in the presence of a test substance and in the absence of the test substance and comparing an amount of the label bound with a guanine nucleotide-binding region under the both conditions.

19. A screening method for a substance having a therapeutic activity against a lifestyle-related disease or cibophobia, which comprises comparing, in each constitution unit of the screening system of claim 16, an activity of the effector in the presence of a test substance and in the absence of the test substance.

20. A method for identifying a G protein α subunit capable of coupling with a polypeptide consisting of an amino acid sequence shown in SEQ ID NO:2, which comprises adding, in each constitution unit of the screening system of claim 13, a labeled GTP analog in the presence of a ligand for said polypeptide and in the absence of said ligand, and comparing an amount of the label bound with a guanine nucleotide-binding region among constitution units.

21. A method for identifying a G protein α subunit capable of coupling with a polypeptide consisting of an amino acid sequence shown in SEQ ID NO:2, which comprises comparing an activity of the effector in each constitution unit of the screening system of claim 16 in the presence of a ligand for said polypeptide and in the absence of said ligand.

22. A screening method for a substance having a therapeutic activity against a lifestyle-related disease or cibophobia, which comprises applying the method of claim 18 or 19 only to a system comprising, as a constituent element, a polypeptide comprising a receptor-binding region of the G protein α subunit as identified by the method of claim 20 or 21.

23. The method of claim 22, wherein the G protein α subunit belongs to a Gs family.

24. A screening system for a ligand for a polypeptide of the following:
(a) a polypeptide consisting of an amino acid sequence shown in SEQ ID NO:2, or
(b) a polypeptide consisting of an amino acid sequence shown in SEQ ID NO:2, wherein one or more amino acids of the amino acid sequence have been substituted, deleted, inserted, added or modified, which shows a ligand—receptor interaction of the same level as the polypeptide of (a), and which is coupled with a G protein α subunit belonging to a Gs family and shows an activity to promote a GDP/GTP exchange reaction of the subunit, which comprises, as constituent elements, a lipid bilayer membrane comprising said polypeptide and a polypeptide comprising at least a receptor-binding region of a G protein α subunit belonging to a Gs family and a guanine nucleotide-binding region of any G protein α subunit.

25. The screening system of claim 24, which comprises an eucaryotic host cell transfected with an expression vector comprising a DNA encoding the polypeptide of (a) or (b), and an expression vector comprising a DNA encoding a polypeptide comprising at least a receptor-binding region of a G protein α subunit belonging to a Gs family and a guanine nucleotide-binding region of any G protein α subunit, a homogenate of said cell or a membrane fraction derived from said cell.

26. The screening system of claim 24, which comprises an eucaryotic host cell transfected with an expression vector comprising a DNA encoding a polypeptide fused with a polypeptide comprising, on a C terminal side of said polypeptide, (a) or (b), at least a receptor-binding region of a G protein α subunit belonging to a Gs family and a guanine nucleotide-binding region of any G protein α subunit, a homogenate of said cell or a membrane fraction derived from said cell.

27. The screening system of claim 24, wherein the polypeptide comprising a receptor-binding region of a G protein α subunit belonging to a Gs family and a guanine nucleotide-binding region of any G protein α subunit further comprises any effector interacting region and the lipid bilayer membrane further comprises an effector that interacts with said region.

28. The screening system of claim 27, wherein the effector is adenyl cyclase.

29. The screening system of claim 24, which is a system for searching a substance having a therapeutic activity against a lifestyle-related disease or cibophobia.

30. The screening system of claim 29, wherein the therapeutic activity against a lifestyle-related disease is a feeding suppressive activity, an anti-obesity activity, an anti-diabetic activity or an anti-hyperlipidemic activity.

31. A screening method for a ligand for a polypeptide of the following:
(a) a polypeptide consisting of an amino acid sequence shown in SEQ ID NO:2, or
(b) a polypeptide consisting of an amino acid sequence shown in SEQ ID NO:2, wherein one or more amino acids of the amino acid sequence have been substituted, deleted, inserted, added or modified, which shows a ligand—receptor interaction of the same level as the polypeptide of (a), and which is coupled with a G protein α subunit belonging to a Gs family and shows an activity to promote a GDP/GTP exchange reaction of the subunit, which comprises adding, in the screening system of claim 24 and in the presence of a test substance and in the absence of the test substance, a labeled GTP analog, and comparing the amount of the label bound with a guanine nucleotide-binding region under the both conditions.

32. A screening method for a ligand for a polypeptide of the following:
(a) a polypeptide consisting of an amino acid sequence shown in SEQ ID NO:2, or
(b) a polypeptide consisting of an amino acid sequence shown in SEQ ID NO:2, wherein one or more amino acids of the amino acid sequence have been substituted, deleted, inserted, added or modified, which shows a ligand—receptor interaction of the same level as the polypeptide of (a), and which is coupled with a G protein α subunit belonging to a Gs family and shows an activity to promote a GDP/GTP exchange reaction of the subunit, which comprises comparing an activity of the effector in the screening system of claim 27 in the presence of a test substance and in the absence of the test substance.

33. The screening method of claim 32, which comprises comparing an amount of cAMP in an eucaryotic host cell in the presence of a test substance and in the absence of the test substance.

34. The screening system of claim 31, which is a system for searching a substance having a therapeutic activity against a lifestyle-related disease or cibophobia.

35. The screening system of any of claims 18, 19, 34, 38, and 39, wherein the therapeutic activity against a lifestyle-related disease is a feeding suppressive activity, an anti-obesity activity, an anti-diabetic activity or an anti-hyperlipidemic activity.

36. A therapeutic agent for a lifestyle-related disease comprising, as an active ingredient, a substance having a therapeutic activity against a lifestyle-related disease, which is obtained by the screening method of any of claims 13-16, 18, 19, 29, 34, 38, and 39.

37. A therapeutic agent for cibophobia comprising, as an active ingredient, a substance having a therapeutic activity against cibophobia, which is obtained by the screening method of any of claims 13-16, 18, 19, 29, 34, 38 and, 39.

38. The screening system of claim 32, which is a system for searching a substance having a therapeutic activity against a lifestyle-related disease or cibophobia.

39. The screening system of claim 33, which is a system for searching a substance having a therapeutic activity against a lifestyle-related disease or cibophobia.

40. The screening system of claim 22, wherein the therapeutic activity against a lifestyle-related disease is a feeding suppressive activity, an anti-obesity activity, an anti-diabetic activity or an anti-hyperlipidemic activity.

41. A therapeutic agent for a lifestyle-related disease comprising, as an active ingredient, a substance having a therapeutic activity against a lifestyle-related disease, which is obtained by the screening method of claim 22.

42. A therapeutic agent for cibophobia comprising, as an active ingredient, a substance having a therapeutic activity against cibophobia, which is obtained by the screening method of claim 22.


SEQUENCE LISTING (25 sequences)

SEQ ID NO: 1 - DNA, 1926 bp, Homo sapiens
  CDS (154)..(1152)
gaauucggcucgagcgcagcagcgcgcagguccucaccagagcucugguggccaccucugucccgccaugcugcucaccgacaguggccagggcccacagcaccaagaggcuugggccacaaaguaaagggucgcggagccucgccggccgccauguggagcugcagcugguucaacggcacagggcugguggaggagcugccugccugccaggaccugcagcuggggcugucacuguugucgcugcugggccugguggugggcgugccagugggccugugcuacaacgcccugcuggugcuggccaaccuacacagcaaggccagcaugaccaugccggacguguacuuugucaacauggcaguggcaggccuggugcucagcgcccuggccccugugcaccugcucggccccccgagcucccggugggcgcuguggagugugggcggcgaaguccacguggcacugcagauccccuucaauguguccucacugguggccauguacuccaccgcccugcugagccucgaccacuacaucgagcgugcacugccgcggaccuacauggccagcguguacaacacgcggcacgugugcggcuucgugugggguggcgcgcugcugaccagcuucuccucgcugcucuucuacaucugcagccauguguccacccgcgcgcuagagugcgccaagaugcagaacgcagaagcugccgacgccacgcugguguucaucggcuacguggugccagcacuggccacccucuacgcgcuggugcuacucucccgcguccgcagggaggacacgccccuggaccgggacacgggccggcuggagcccucggcacacaggcugcugguggccaccgugugcacgcaguuugggcucuggacgccacacuaucugauccugcuggggcacacggucaucaucucgcgagggaagcccguggacgcacacuaccuggggcuacugcacuuugugaaggauuucuccaaacuccuggccuucuccagcagcuuugugacaccacuucucuaccgcuacaugaaccagagcuuccccagcaagcuccaacggcugaugaaaaagcugcccugcggggaccggcacugcuccccggaccacaugggggugcagcaggugcuggcguaggcggcccagcccuccuggggagacgugacucugguggacgcagagcacuuaguuacccuggacgcuccccacauccuuccagaaggagacgagcugcuggaagagaagcaggagggguguuuuucuugaaguuuccuuuuucccacaaaugccacucuugggccaaggcugugguccccguggcuggcaucuggcuugagucuccccgaggccugugcgucucccaaacacgcagcucaagguccacauccgcaaaagccuccucgccuucagccuccucagcauucaguuugucaaugaagugaugaaagcuuagagccaguauuuauacuuugugguuaaaauacuugauucccccuuguuuguuuuacaaaaacagauguuuccuagaaaaaugacaaauaguaaaaugaacaaaacccuacgaaagaauggcaacagccaggguggccgggcccugccagugggcggcgugugcuagcaaggccugccgggugugccgcagucaccacaggguucugagaacauuucacagaagugccugagacgcggagacauggcugguguuaaauggagcuauucaauagcagugacgcgcucuccucagccaccaaaugucccugacacccuccccagcccccacagauaacaucagcugagguuuuuuucaguaugaaccuguccuaaaucaauuccucaaagugugcacaaaacuaaagaauauaaauaaacaaaagaaaggugaaaaaaaaaaaaaaaaaagggcggccg

SEQ ID NO: 2 - protein, 333 aa, Homo sapiens
MWSCSWFNGTGLVEELPACQDLQLGLSLLSLLGLVVGVPVGLCYNALLVLANLHSKASMTMPDVYFVNMAVAGLVLSALAPVHLLGPPSSRWALWSVGGEVHVALQIPFNVSSLVAMYSTALLSLDHYIERALPRTYMASVYNTRHVCGFVWGGALLTSFSSLLFYICSHVSTRALECAKMQNAEAADATLVFIGYVVPALATLYALVLLSRVRREDTPLDRDTGRLEPSAHRLLVATVCTQFGLWTPHYLILLGHTVIISRGKPVDAHYLGLLHFVKDFSKLLAFSSSFVTPLLYRYMNQSFPSKLQRLMKKLPCGDRHCSPDHMGVQQVLA

SEQ ID NO: 3 - DNA, 25 bp, Artificial
  Oligonucleotide designed to act as an antisense DNA inhibiting expression of clone 901.
gcttcgcagcgctcgctgggcggcg

SEQ ID NO: 4 - DNA, 25 bp, Artificial
  Oligonucleotide designed to act as an antisense DNA inhibiting expression of clone 901.
agtgggccacagctccacatggcag

SEQ ID NO: 5 - DNA, 20 bp, Artificial
  Oligonucleotide designed to act as sense primer for amplifying human G protein G-alpha-16 cDNA fragment containing full length ORF.
ccatggcccgctcgctgacc

SEQ ID NO: 6 - DNA, 21 bp, Artificial
  Oligonucleotide designed to act as antisense primer for amplifying human G protein G-alpha-16 cDNA fragment containing full length ORF.
ccgaggctggagagatagacc

SEQ ID NO: 7 - DNA, 19 bp, Artificial
  Oligonucleotide designed to act as sense primer for amplifying human G protein G-alpha-i2 cDNA fragment containing full length ORF.
gcggcggagcggcggaacg

SEQ ID NO: 8 - DNA, 24 bp, Artificial
  Oligonucleotide designed to act as antisense primer for amplifying human G protein G-alpha-i2 cDNA fragment containing full length ORF.
ggagaaaagcggcgggggaacagg

SEQ ID NO: 9 - DNA, 21 bp, Artificial
  Oligonucleotide designed to act as sense primer for amplifying human G protein G-alpha-S2 cDNA fragment containing full length ORF.
ccatgggctgcctcgggaaca

SEQ ID NO: 10 - DNA, 23 bp, Artificial
  Oligonucleotide designed to act as antisense primer for amplifying human G protein G-alpha-S2 cDNA fragment containing full length ORF.
ggtttcgcaaaatcactcggggg

SEQ ID NO: 11 - DNA, 21 bp, Artificial
  Oligonucleotide designed to act as sense primer for amplifying human G protein G-alpha-16 cDNA fragment from initiation codon.
atggcccgctcgctgacctgg

SEQ ID NO: 12 - DNA, 21 bp, Artificial
  Oligonucleotide designed to act as sense primer for amplifying human G protein G-alpha-i2 cDNA fragment from initiation codon.
atgggctgcaccgtgagcgcc

SEQ ID NO: 13 - DNA, 20 bp, Artificial
  Oligonucleotide designed to act as sense primer for amplifying human G protein G-alpha-S2 cDNA fragment from initiation codon.
atgggctgcctcgggaacag

SEQ ID NO: 14 - DNA, 18 bp, Artificial
  Oligonucleotide designed to act as antisense primer for amplifying multiple cloning site of plasmid pcDNA3.1(+).
tagaaggcacagtcgagg

SEQ ID NO: 15 - DNA, 24 bp, Artificial
  Sense strand oligonucleotide designed to construct linker containing nucleotide sequence encoding 6xHis-tag peptide sequence.
gatatccatcatcatcatcaccat

SEQ ID NO: 16 - DNA, 18 bp, Artificial
  Antisense strand oligonucleotide desined to construct linker containing nucleotide sequence encoding 6xHis-tag peptide sequence.
atggtgatgatgatgatg

SEQ ID NO: 17 - DNA, 21 bp, Artificial
  Oligonucleotide designed to act as sense primer for amplifying human clone 901 mRNA containing full length ORF.
cctcgccggccgccatgtgga

SEQ ID NO: 18 - DNA, 20 bp, Artificial
  Oligonucleotide designed to act as antisense primer for amplifying human clone 901 mRNA up to immediately before stop codon.
cgccagcacctgctgcaccc

SEQ ID NO: 19 - DNA, 20 bp, Artificial
  Oligonucleotide designed to act as antisense primer for amplifying human clone 901 mRNA containing full length ORF.
gagcgtccagggtaactaag

SEQ ID NO: 20 - DNA, 2258 bp, Artificial
  Insert cDNA sequence contained in pc901HISG- alpha-16.
cctcgccggccgccatgtggagctgcagctggttcaacggcacagggctggtggaggagctgcctgcctgccaggacctgcagctggggctgtcactgttgtcgctgctgggcctggtggtgggcgtgccagtgggcctgtgctacaacgccctgctggtgctggccaacctacacagcaaggccagcatgaccatgccggacgtgtactttgtcaacatggcagtggcaggcctggtgctcagcgccctggcccctgtgcacctgctcggccccccgagctcccggtgggcgctgtggagtgtgggcggcgaagtccacgtggcactgcagatccccttcaatgtgtcctcactggtggccatgtactccaccgccctgctgagcctcgaccactacatcgagcgtgcactgccgcggacctacatggccagcgtgtacaacacgcggcacgtgtgcggcttcgtgtggggtggcgcgctgctgaccagcttctcctcgctgctcttctacatctgcagccatgtgtccacccgcgcgctagagtgcgccaagatgcagaacgcagaagctgccgacgccacgctggtgttcatcggctacgtggtgccagcactggccaccctctacgcgctggtgctactctcccgcgtccgcagggaggacacgcccctggaccgggacacgggccggctggagccctcggcacacaggctgctggtggccaccgtgtgcacgcagtttgggctctggacgccacactatctgatcctgctggggcacacggtcatcatctcgcgagggaagcccgtggacgcacactacctggggctactgcactttgtgaaggatttctccaaactcctggccttctccagcagctttgtgacaccacttctctaccgctacatgaaccagagcttccccagcaagctccaacggctgatgaaaaagctgccctgcggggaccggcactgctccccggaccacatgggggtgcagcaggtgctggcgatccatcatcatcatcaccatatggcccgctcgctgacctggcgctgctgcccctggtgcctgacggaggatgagaaggccgccgcccgggtggaccaggagatcaacaggatcctcttggagcagaagaagcaggaccgcggggagctgaagctgctgcttttgggcccaggcgagagcgggaagagcaccttcatcaagcagatgcggatcatccacggcgccggctactcggaggaggagcgcaagggcttccggcccctggtctaccagaacatcttcgtgtccatgcgggccatgatcgaggccatggagcggctgcagattccattcagcaggcccgagagcaagcaccacgctagcctggtcatgagccaggacccctataaagtgaccacgtttgagaagcgctacgctgcggccatgcagtggctgtggagggatgccggcatccgggcctgctatgagcgtcggcgggaattccacctgctcgattcagccgtgtactacctgtcccacctggagcgcatcaccgaggagggctacgtccccacagctcaggacgtgctccgcagccgcatgcccaccactggcatcaacgagtactgcttctccgtgcagaaaaccaacctgcggatcgtggacgtcgggggccagaagtcagagcgtaagaaatggatccattgtttcgagaacgtgatcgccctcatctacctggcctcactgagtgaatacgaccagtgcctggaggagaacaaccaggagaaccgcatgaaggagagcctcgcattgtttgggactatcctggaactaccctggttcaaaagcacatccgtcatcctctttctcaacaaaaccgacatcctggaggagaaaatccccacctcccacctggctacctatttccccagtttccagggccctaagcaggatgctgaggcagccaagaggttcatcctggacatgtacacgaggatgtacaccgggtgcgtggacggccccgagggcagcaagaagggcgcacgatcccgacgcctcttcagccactacacatgtgccacagacacacagaacatccgcaaggtcttcaaggacgtgcgggactcggtgctcgcccgctacctggacgagatcaacctgctgtgacccaggccccacctggggcaggcggcaccggcgggcgggtgggaggtgggagtggctgcagggacccctagtgtccctggtctatctctccagcctcgg

SEQ ID NO: 21 - protein, 714 aa, Artificial
  Insert cDNA sequence contained in pc901HISG- alpha-16.
MWSCSWFNGTGLVEELPACQDLQLGLSLLSLLGLVVGVPVGLCYNALLVLANLHSKASMTMPDVYFVNMAVAGLVLSALAPVHLLGPPSSRWALWSVGGEVHVALQIPFNVSSLVAMYSTALLSLDHYIERALPRTYMASVYNTRHVCGFVWGGALLTSFSSLLFYICSHVSTRALECAKMQNAEAADATLVFIGYVVPALATLYALVLLSRVRREDTPLDRDTGRLEPSAHRLLVATVCTQFGLWTPHYLILLGHTVIISRGKPVDAHYLGLLHFVKDFSKLLAFSSSFVTPLLYRYMNQSFPSKLQRLMKKLPCGDRHCSPDHMGVQQVLAIHHHHHHMARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLQIPFSRPESKHHASLVMSQDPYKVTTFEKRYAAAMQWLWRDAGIRACYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNIRKVFKDVRDSVLARYLDEINLL

SEQ ID NO: 22 - DNA, 2216 bp, Artificial
  Insert cDNA sequence contained in pc901HISG- alpha-i2.
cctcgccggccgccatgtggagctgcagctggttcaacggcacagggctggtggaggagctgcctgcctgccaggacctgcagctggggctgtcactgttgtcgctgctgggcctggtggtgggcgtgccagtgggcctgtgctacaacgccctgctggtgctggccaacctacacagcaaggccagcatgaccatgccggacgtgtactttgtcaacatggcagtggcaggcctggtgctcagcgccctggcccctgtgcacctgctcggccccccgagctcccggtgggcgctgtggagtgtgggcggcgaagtccacgtggcactgcagatccccttcaatgtgtcctcactggtggccatgtactccaccgccctgctgagcctcgaccactacatcgagcgtgcactgccgcggacctacatggccagcgtgtacaacacgcggcacgtgtgcggcttcgtgtggggtggcgcgctgctgaccagcttctcctcgctgctcttctacatctgcagccatgtgtccacccgcgcgctagagtgcgccaagatgcagaacgcagaagctgccgacgccacgctggtgttcatcggctacgtggtgccagcactggccaccctctacgcgctggtgctactctcccgcgtccgcagggaggacacgcccctggaccgggacacgggccggctggagccctcggcacacaggctgctggtggccaccgtgtgcacgcagtttgggctctggacgccacactatctgatcctgctggggcacacggtcatcatctcgcgagggaagcccgtggacgcacactacctggggctactgcactttgtgaaggatttctccaaactcctggccttctccagcagctttgtgacaccacttctctaccgctacatgaaccagagcttccccagcaagctccaacggctgatgaaaaagctgccctgcggggaccggcactgctccccggaccacatgggggtgcagcaggtgctggcgatccatcatcatcatcaccatatgggctgcaccgtgagcgccgaggacaaggcggcggccgagcgctctaagatgatcgacaagaacctgcgggaggacggagagaaggcggcgcgggaggtgaagttgctgctgttgggtgctggggagtcagggaagagcaccatcgtcaagcagatgaagatcatccacgaggatggctactccgaggaggaatgccggcagtaccgggcggttgtctacagcaacaccatccagtccatcatggccattgtcaaagccatgggcaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctgaggggcagcggggcctggcgggatgggccaccgccgactttgtaccccccaacccctgaggaagatgggggcaagaagatcacgctccccgcctgttcccccgccgcttttctcc

SEQ ID NO: 23 - protein, 695 aa, Artificial [sequence omitted]

SEQ ID NO: 24 - DNA, 2363 bp, Artificial
  Insert cDNA sequence contained in pc901HISG- alpha-S2.
cctcgccggccgccatgtggagctgcagctggttcaacggcacagggctggtggaggagctgcctgcctgccaggacctgcagctggggctgtcactgttgtcgctgctgggcctggtggtgggcgtgccagtgggcctgtgctacaacgccctgctggtgctggccaacctacacagcaaggccagcatgaccatgccggacgtgtactttgtcaacatggcagtggcaggcctggtgctcagcgccctggcccctgtgcacctgctcggccccccgagctcccggtgggcgctgtggagtgtgggcggcgaagtccacgtggcactgcagatccccttcaatgtgtcctcactggtggccatgtactccaccgccctgctgagcctcgaccactacatcgagcgtgcactgccgcggacctacatggccagcgtgtacaacacgcggcacgtgtgcggcttcgtgtggggtggcgcgctgctgaccagcttctcctcgctgctcttctacatctgcagccatgtgtccacccgcgcgctagagtgcgccaagatgcagaacgcagaagctgccgacgccacgctggtgttcatcggctacgtggtgccagcactggccaccctctacgcgctggtgctactctcccgcgtccgcagggaggacacgcccctggaccgggacacgggccggctggagccctcggcacacaggctgctggtggccaccgtgtgcacgcagtttgggctctggacgccacactatctgatcctgctggggcacacggtcatcatctcgcgagggaagcccgtggacgcacactacctggggctactgcactttgtgaaggatttctccaaactcctggccttctccagcagctttgtgacaccacttctctaccgctacatgaaccagagcttccccagcaagctccaacggctgatgaaaaagctgccctgcggggaccggcactgctccccggaccacatgggggtgcagcaggtgctggcgatccatcatcatcatcaccatatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggcagtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacc

SEQ ID NO: 25 - protein, 735 aa, Artificial [sequence omitted]